(19) United States
(12) Patent Application Publication
(10) Pub. No.: US 2025/0031644 A1
(43) Pub. Date: Jan. 30, 2025

(54) MODIFIED GENE FOR MORE EFFICIENT CULTIVATION OF CUCUMBER

(71) Applicant: RIJK ZWAAN ZAADTEELT EN ZAADHANDEL B.V., De Lier (NL)

(72) Inventors: Magdalena Barbara LASTDRAGER, De Lier (NL); Taco Peter JESSE, De Lier (NL); Adrianus Cornelis KOEKEN, De Lier (NL); Cornelis HAARING, De Lier (NL)

(21) Appl. No.: 18/797,867

(22) Filed: Aug. 8, 2024

Related U.S. Application Data
(63) Continuation-in-part of application No. PCT/EP2023/053276, filed on Feb. 10, 2023.

(30) Foreign Application Priority Data
Feb. 10, 2022 (WO) PCT/EP2022/053284

(51) Int. Cl. A01H 1/00; A01H 1/04; A01H 6/34

(52) U.S. Cl. CPC A01H 1/121; A01H 1/045; A01H 6/346

(57) ABSTRACT

The present invention relates to a modified DCAF8 gene, the wild type of which gene having a coding sequence according to SEQ ID No. 2, encoding the protein of SEQ ID No. 3, or having a coding sequence and a protein sequence that show at least 70% sequence identity to SEQ ID No. 2 and at least 70% sequence similarity to SEQ ID No. 3, respectively, and wherein the modified DCAF8 gene comprises one or more nucleotides replaced, inserted and/or deleted relative to the wild type, and wherein the modified DCAF8 gene when present homozygously, confers to a cucurbit plant a reduced leaf area phenotype as compared to a cucurbit plant not having the modified gene. The invention further relates to a plant comprising the modified DCAF8 gene of the invention.

### RELATED APPLICATIONS AND INCORPORATION BY REFERENCE

This application is a continuation-in-part of copending International Patent Application No. PCT/EP2023/053276, filed Feb. 10, 2023, and published as International Publication No. WO 2023/152274 on Aug. 17, 2023 and which claims priority to International Application No. PCT/EP2022/053284, filed Feb. 10, 2022, the contents of which are incorporated by reference herein in their entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created on Jul. 30, 2024, is named Y7954-00626SL.xml, and is 31,874 bytes in size.

### FIELD OF THE INVENTION

The present invention relates to a modified plant gene in cucumber (Cucumis sativus L.) and other species of the Cucurbitaceae plant family that leads to a reduced leaf area phenotype in plants comprising said gene. The invention further relates to methods for identifying, selecting and producing plants comprising said modified gene. The invention also relates to a marker for identifying plants comprising the modified gene and the use of said marker.

### BACKGROUND OF THE INVENTION

The cucurbit family (Cucurbitaceae) includes several economically important cultivated plants, such as cucumber (Cucumis sativus L.), melon (Cucumis melo L.), watermelon (Citrullus lanatus (Thunb.) Matsum. & Nakai) and squashes/pumpkins (Cucurbita spp.), together referred to as cucurbits.

In the breeding of cucurbits, the most important traits are fruit quality (e.g. less bitterness), plant vigour, disease resistance and yield. Yield increase is amongst the most complex quantitative traits to acquire.

It was an object of the research work preceding the present invention to tackle the difficult problem of yield increase in cucurbits.

Overall, yield research seems to be hindered by the fact that the genetic base of most of the presently cultivated cucurbits is rather narrow and offers limited room for recombination and improvement, especially in cucumber. It was contemplated by the present inventors that an increase in yield per area can be achieved by increasing the efficiency of cultivation. Higher efficiency in plant cultivation can be attained by using plants exhibiting special plant architecture, such as plants characterized by smaller leaf area and shorter internodes, as they require less growing area. Thus, plants exhibiting special plant architecture can be planted at higher densities, provided that the change in plant architecture has no negative effect on the yield.

The size of plant organs, such as leaf and fruit, is under tight genetic control. Various genes have been identified that regulate plant organ size. The most well studied is the STERILE APETALA (SAP) in Arabidopsis. The SAP gene is known to encode an F-box protein which is a component of the SKP1/Cullin/F-box E3 ubiquitin ligase complex (Wang et al., Nat. Commun. 7, 11192). SAP was found to modulate the stability of a repressor protein complex by targeting the KIX-PPD repressor complex for proteasomal degradation [Li et al., PLoS Genet 14(2)]. The orthologous gene in cucumber is called LITTLE LEAF (LL) [Yang et al., The Plant J., 95(5)], which encodes a WD40 repeat domain-containing protein. LL was shown to be an important player in organ size control and lateral branch development in cucumber. A mutated allele of LL, the ll allele, was found in various cucumber accessions having smaller organ sizes and more lateral branches.

In the research work that led to the present invention, a new genetic determinant responsible for reduced leaf area was identified in C. sativus var. hardwickii, and further confirmed in other species belonging to the Cucurbitaceae family. The research work revealed that the reduced leaf area is related to a specific mutation in the DDB1- and CUL4-associated factor 8 (DCAF8) gene. The DCAF8 gene encodes the DDB1- and CUL4-associated factor 8 protein (DCAF8), which functions as a substrate-recognition receptor for CUL4-DDB1 E3 ubiquitin ligase, linked to protein degradation.

Protein degradation is a major aspect of eukaryotic cell regulation. The main mechanism of protein degradation involves the tagging of the target protein by a small protein called ubiquitin, in the ubiquitination pathway. E3 ligases form part of this three-enzyme (E1-E2-E3) pathway. The selectivity of the protein degradation system largely depends on the great number of E3 ligases. There are at least six classes of E3 ligases in plants, one of them is the class of CUL4-DDB1 E3 ubiquitin ligases. This class of ligases uses the triple beta propeller-forming DDB1 (DNA DAMAGED BINDING 1) and a secondary protein (DCAF, for DDB1- and CUL4-associated factor) to bind the target protein. CUL4-DDB1 E3 ubiquitin ligases are involved in various aspects of plant development and physiology, as well as the regulation of photomorphogenesis.

DCAF proteins are ubiquitous in eukaryotes and are abundantly present in plants (they have 119 members in Arabidopsis). Structurally, DCAF proteins are characterized by the presence of multiple WD40 repeats that fold around a central axis into a propeller-like structure and serve as the substrate-recruiting module of the protein. DCAF8 is characterized by 7 WD40 repeats, as well as a conserved WD×R motif and a DWD box, the latter two facilitating the interaction with DDB1.

In the publicly available genome assembly of Cucumis sativus L. var. sativus cv. 9930 version 2 [Huang S et al. (2009) The genome of the cucumber, Cucumis sativus L. Nature Genetics 41:1275-1281], the wild type DCAF8 gene in Cucumis sativus is located on chromosome 7, between positions 947172 and 954937.

Citation or identification of any document in this application is not an admission that such document is available as prior art to the present invention.

### SUMMARY OF THE INVENTION

The object of the invention has been achieved by providing a modified DCAF8 gene which, when expressed in a plant, leads to a plant having smaller leaves than a plant lacking the modified gene, making it suitable for planting at higher densities.

According to one aspect of the present invention, the invention provides for a modified DCAF8 gene, the wild type of which gene having a coding sequence according to SEQ ID No. 2, encoding the protein of SEQ ID No. 3, or having a coding sequence and a protein sequence that show at least 70% sequence identity to SEQ ID No. 2 and at least 70% sequence similarity to SEQ ID No. 3, respectively, and wherein the modified DCAF8 gene comprises one or more nucleotides replaced, inserted and/or deleted relative to the wild type, and wherein the modified DCAF8 gene when present homozygously, confers to a cucurbit plant a reduced leaf area phenotype as compared to a cucurbit plant not having the modified gene.

In some embodiments, the modified DCAF8 gene comprises a single nucleotide substitution leading to a missense mutation, on position 592 in SEQ ID No. 2, or on a corresponding position of a homologous sequence having at least 70% sequence identity to SEQ ID No. 2.

In some embodiments, the modified DCAF8 gene comprises a mutation that leads to the substitution Gln->Glu on position 198 of SEQ ID No. 3 or on a corresponding position of a homologous amino acid sequence having at least 70% sequence similarity to SEQ ID No. 3.

According to another aspect of the present invention, the invention provides for a plant comprising the modified DCAF8 gene as described herein.

In some embodiments, the plant comprises the modified DCAF8 gene homozygously, which results in a reduction in leaf area of at least 10% as compared to a plant that is isogenic but does not possess the DCAF8 gene. In some embodiments, the leaf area is the average leaf area of two fully-grown intact leaves of individual plants, and wherein the fully grown leaves are chosen from the 12^{th} to 16^{th} (leaf 1) and the 16^{th} to 19^{th} (leaf 2) leaves of each plant.

In some embodiments, the plant is a cucurbit plant. In some embodiments, the plant is a Cucumis sativus plant.

In some embodiments, the plant is an agronomically elite plant, in particular a hybrid variety or an inbred line.

In some embodiments, the modified gene is as present in a cucumber plant representative seed of which plant is deposited under deposit number NCIMB 43528.

According to another aspect of the present invention, the invention provides for a seed capable of growing into the plant as described herein.

According to another aspect of the present invention, the invention provides for a fruit harvested from the plant as described herein, wherein the fruit comprises the modified gene as described herein.

According to another aspect of the present invention, the invention provides for propagation material suitable for producing the plant as described herein, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the modified DCAF8 gene as described herein.

According to another aspect of the present invention, the invention provides for a use of the modified DCAF8 gene as described herein for producing a plant exhibiting a reduced leaf area phenotype.

According to another aspect of the present invention, the invention provides for a marker for the identification of the modified DCAF8 gene as described herein, wherein the marker detects a single nucleotide substitution from cytosine to guanine, on position 592 in SEQ ID No. 2, or wherein the marker detects said substitution on a corresponding position of a homologous sequence that has 70% sequence identity to SEQ ID No. 2.

According to another aspect of the present invention, the invention provides for a marker for a use of the marker as described herein for the identification of a plant exhibiting a reduced leaf area phenotype.

According to another aspect of the present invention, the invention provides for a method for selecting the plant exhibiting a reduced leaf area phenotype, comprising identifying the presence of a modification in the DCAF8 gene as herein described, optionally testing the plant for reduced leaf area phenotype, and selecting a plant comprising the modified DCAF8 gene.

According to another aspect of the present invention, the invention provides for a method for producing a plant exhibiting the reduced leaf area phenotype as described herein, comprising the step of introducing a mutation into the DCAF8 gene, such that a protein product of the mutated DCAF8 gene leads to a reduced leaf area phenotype in the plant. In some embodiments, the mutation is as described herein.

According to another aspect of the present invention, the invention provides for a method for producing a plant exhibiting reduced leaf area phenotype comprising the steps of:
a) crossing a first parent plant comprising the modified DCAF8 gene as described herein with a second parent plant to obtain an F1 population;
b) optionally performing one or more rounds of selfing and/or crossing with a plant from the F1 population to obtain a further generation population;
c) selecting from the population a plant that comprises the modified DCAF8 gene homozygously, as a plant exhibiting reduced leaf area phenotype.

According to another aspect of the present invention, the invention provides for a method for producing hybrid seed, comprising the steps of crossing a first parent plant with a second parent plant, wherein one or both parent plants are homozygous for the modified DCAF8 gene as described herein, and harvesting the hybrid seed.

According to another aspect of the present invention, the invention provides for a hybrid seed as produced by the method as described herein.

According to another aspect of the present invention, the invention provides for a plant that is grown from the hybrid seed as described herein.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposits

Seed of Cucumis sativus L. comprising the modified DCAF8 gene of the invention was deposited with NCIMB Ltd, Ferguson Building, Craibstone Estate, Bucksburn, Aberdeen AB21 9YA, UK on 9 Mar. 2020, under deposit accession numbers NCIMB 43586 and was made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Sequence alignment of the genomic DNA sequences for DCAF8 in various species of the Cucurbitaceae family (Cmo—Cucurbita moschata—, Cmx—Cucurbita maxima, Cp—Cucurbita pepo subsp. pepo, Cl—Citrullus lanatus, Cs—Cucumis sativus, Cm—Cucumis melo).

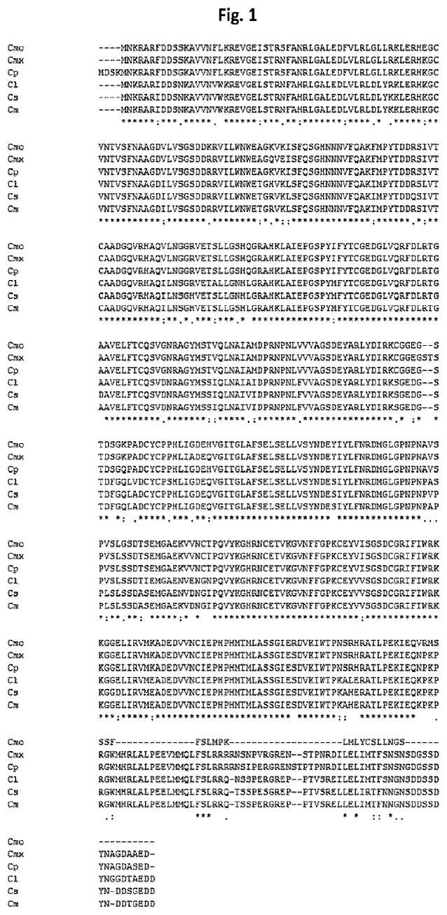

### DETAILED DESCRIPTION OF THE INVENTION

The present invention provides a modified DCAF8 gene, the wild type of which having a genomic sequence according to SEQ ID No. 1 and a coding sequence according to SEQ ID No. 2, or a genomic sequence and coding sequence that in order of increased preference, has 70%, 75%, 80%, 85%, 90%, 91% 92%, 93%, 94%, 95%, 96%, 97%, 98%, 99% sequence identity to SEQ ID No. 1 or SEQ ID No. 2, respectively, and wherein SEQ ID No. 2 encodes a protein having SEQ ID No. 3, or a protein having in order of increased preference at least 70%, 75%, 80%, 85%, 90%, 91% 92%, 93%, 94%, 95%, 96%, 97%, 98%, 99% sequence similarity to SEQ ID No. 3, and wherein the modified DCAF8 gene comprises one or more nucleotides replaced, inserted and/or deleted relative to the wild type, and wherein said one or more replaced, inserted and/or deleted nucleotides result in a modification of the DCAF8 protein function.

As used herein, sequence identity or sequence similarity is the percentage of nucleotides or amino acids that is identical or similar between two sequences after proper alignment of those sequences. The person skilled in the art is aware of how to align sequences, for example by using a sequence alignment tool such as BLAST, which can be used for both nucleotide sequences and protein sequences. To obtain the most significant result, the best possible alignment that gives the highest sequence identity or similarity score should be obtained. The percentage sequence identity or similarity is calculated through comparison over the length of the shortest sequence in the assessment, whereby in the present case a sequence that is included in such assessment represents a gene that at least comprises a start codon and a stop codon, or a complete protein encoded by such a gene. Sequence identity is used for comparison of nucleotide sequences. Sequence similarity is used to compare amino acid sequences, whereby conservative amino acid substitutions are deemed to be similar and is calculated herein based on the BLOSUM62 scoring matrix.

The modified DCAF8 gene of the invention comprises one or more nucleotides replaced, inserted and/or deleted relative to the wild type, and said one or more replaced, inserted and/or deleted nucleotides result in a modified functionality of the DCAF8 protein. The modification in the DCAF8 protein functionality can have but is not limited to one of the following causes: i) the DCAF8 protein functionality can be altered as a result of a modification in the biochemical properties of the protein, due to a change in the amino acids sequence; ii) for instance, the DCAF8 protein functionality can be altered as a result of the replacement of an uncharged amino acid by a negatively charged amino acid in the vicinity of two conserved regions; iii) specifically, the DCAF8 protein functionality can be altered as a result of the replacement of an uncharged glutamine, situated inside the protein, by a negatively charged glutamate, situated on the surface of the protein; iv) more specifically, the DCAF8 protein functionality can be altered as a result of a loss of a nucleotide binding site, due to changes in the secondary structure of the protein.

Therefore, the present invention provides a modified DCAF8 gene wherein the modified gene comprises a mutation in SEQ ID No. 2, or in a homologous sequence having in order of increased preference at least 70%, 75%, 80%, 85%, 90%, 91% 92%, 93%, 94%, 95%, 96%, 97%, 98%, 99% sequence identity to SEQ ID No. 2, said mutation leading to a modification in protein functionality due to a non-conservative amino acid replacement in SEQ ID No. 3 or in a homologous amino acid sequence having in order of increased preference at least 70%, 75%, 80%, 85%, 90%, 91% 92%, 93%, 94%, 95%, 96%, 97%, 98%, 99% sequence similarity to SEQ ID No. 3.

In one embodiment, the present invention provides a modified DCAF8 gene, wherein the modified gene comprises a mutation caused by the replacement of a cytosine by a guanine at nucleotide position 2538 in SEQ ID No. 1, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID No. 1, or wherein the modified DCAF8 gene comprises a mutation caused by the replacement of a cytosine by a guanine at position 592 in SEQ ID No. 2, or in a homologous sequence having in order of increased preference at least 70%, 75%, 80%, 85%, 90%, 91% 92%, 93%, 94%, 95%, 96%, 97%, 98%, 99% sequence identity to SEQ ID No. 2, or wherein the modified DCAF8 gene encodes a protein having a non-conservative amino acid replacement on position 198 of SEQ ID No. 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence similarity to SEQ ID No. 3.

In a particular aspect, the present invention provides a modified DCAF8 gene, wherein the modification is a missense mutation, leading to an amino acid replacement of a glutamine to glutamate on position 198 of SEQ ID No. 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence similarity to SEQ ID No. 3.

In another particular aspect, the modified DCAF8 gene of the invention comprises a genomic sequence having SEQ ID No. 4, or a coding sequence having SEQ ID No. 5, or a sequence encoding a protein having SEQ ID No. 6.

In a further aspect, the invention relates to a modified DCAF8 gene comprising a missense mutation, wherein the missense mutation leads to the replacement of an uncharged glutamine into a charged glutamate, influencing the secondary structure of the protein, and leading to the loss of a nucleic acid binding site.

The modified DCAF8 gene of the invention, when present homozygously in a plant, in particular in a plant of the Cucurbitaceae plant family and more in particular in a cucumber plant, leads to a phenotype characterized by reduced leaf area. Thus, the ‘gene of the invention’ is the modified DCAF8 gene, which can be present either homozygously or heterozygously in a plant. The modified DCAF8 gene, independently of whether it is present homozygously or heterozygously in a plant, is a gene of the invention.

The invention further relates to a plant, preferably a plant of the Cucurbitaceae plant family, more preferably a plant belonging to the Cucumis genus, even more preferably a Cucumis sativus plant, wherein the plant comprises the modified DCAF8 gene as described in the present application in its genome. A plant conform to the above description is referred to herein as a ‘plant of the invention’.

In one embodiment, the plant of the invention is an agronomically elite plant, preferably an agronomically elite plant of the Cucurbitaceae family, most preferably a Cucumis sativus plant.

In the context of this invention, an agronomically elite plant is a plant having a genotype that, as a result of human intervention, comprises an accumulation of distinguishable and desirable agronomic traits which allow a producer to harvest a product of commercial significance. Preferably the agronomically elite plant of the invention is a plant of an inbred line or a hybrid.

As used herein, a plant of an inbred line is a plant of a population of plants that is the result of three or more rounds of selfing, or backcrossing; or which plant is a doubled haploid. An inbred line may e.g., be a parent line used for the production of a commercial hybrid.

As used herein, a hybrid plant is a plant which is the result of a cross between two different plants having different genotypes. More in particular, a hybrid plant is the result of a cross between plants of two different inbred lines, such a hybrid plant may e.g. be a plant of an F1 hybrid variety.

Seed of Cucumis sativus L. comprising the modified DCAF8 gene of the invention homozygously was deposited with the NCIMB under accession number NCIMB 43528.

The invention thus relates to plants grown from seed deposited under NCIMB accession number NCIMB 43528.

As used herein, the presence of the reduced leaf area phenotype is determined by measuring the leaf area. When comparing leaf area of plants having the modified gene with plants that do not have the modified gene, it is required that the leaf area is measured in a similar way for both types of plants. Leaf area measurements are done on two fully-grown intact leaves of individual plants, where leaves are taken from the same leaf height (e.g. the 16th and 19th leaves) during a particular measurement. The leaf area is measured on the leaf blade, excluding the petiole or any other part of the plant. Thus, the term “leaf area” refers to the area of the leaf blade.

The term “reduced leaf area” as used herein is the leaf area that displays a reduction in the leaf area of individual plants of at least 10%, at least 15%, at least 20%, at least 25%, at least 30%, as a result of the homozygous presence of the modified gene of the invention.

To investigate the influence of the gene of the invention on the smaller leaf area, a skilled person has to compare plants of the invention (plants having the gene of the invention) with plants that are isogenic to the plants of the invention but do not possess the gene of the invention. A plant comprising the modified DCAF8 gene of the invention homozygously will typically show a reduction of the leaf area of about 10-30%, when compared to a plant not possessing the modified gene of the invention, wherein the leaf area is obtained by calculating the average leaf area of two fully-grown intact leaves of individual Cucumis sativus plants, and wherein the fully grown leaves are chosen from the 12^{th} to 16^{th} (leaf 1) and the 16^{th} to 19^{th} (leaf 2) leaves of each plant.

In one embodiment, the invention relates to a seed capable of growing into a plant of the invention wherein said plant comprises the modified DCAF8 gene of the invention, in either homozygous or heterozygous state. The invention also relates to the use of said seed for the production of a plant of the invention, by growing said seed into a plant.

A plant comprising the modified gene heterozygously is also a plant of the invention. The plant is a source of the gene.

Yet another embodiment of the invention relates to a fruit harvested from a plant of the invention wherein said fruit comprises the modified DCAF8 gene of the invention, in either homozygous or heterozygous state.

The invention also relates to propagation material suitable for producing a plant of the invention, wherein the propagation material is suitable for sexual reproduction, and is in particular selected from a microspore, a pollen, an ovary, an ovule, an embryo sac and an egg cell, or is suitable for vegetative reproduction, and is in particular selected from a cutting, a root, a stem a cell, and a protoplast, or is suitable for tissue culture of regenerable cells or protoplasts, and is in particular selected from a leaf, a pollen, an embryo, a cotyledon, a hypocotyl, a meristematic cell, a root, a root tip, an anther, a flower, a seed and a stem, wherein the propagation material comprises the modified DCAF8 gene of the invention.

The invention further relates to a cell of a plant of the invention. Such a cell may either be in isolated form or a part of the complete plant or parts thereof and still forms a cell of the invention because such a cell comprises the modified DCAF8 gene of the invention. Each cell of a plant of the invention carries the modified DCAF8 gene of the invention. A cell of the invention may also be a regenerable cell that can regenerate into a new plant of the invention.

The invention further relates to plant tissue of a plant of the invention, which comprises the modified DCAF8 gene. The tissue can be undifferentiated tissue or already differentiated tissue. Undifferentiated tissue is for example a stem tip, an anther, a petal, or pollen, and can be used in micro propagation to obtain new plantlets that are grown into new plants of the invention. The tissue can also be grown from a cell of the invention.

The invention further relates to a method for the production of a plant comprising the modified DCAF8 gene of the invention, which plant shows a reduced leaf area phenotype, by using tissue culture or by using vegetative propagation.

The invention moreover relates to the progeny of a plant, a cell, a tissue, or a seed of the invention, which progeny comprises the modified DCAF8 gene of the invention. Such progeny can in itself be a plant, a cell, a tissue, or a seed. The progeny can in particular be progeny of a plant of the invention deposited under NCIMB number 43528. As used herein, the progeny comprises the first and all further descendants from a cross with a plant of the invention, wherein a cross comprises a cross with itself or a cross with another plant, and wherein a descendant that is determined to be progeny comprises the modified DCAF8 gene of the invention. Descendants can be obtained through selfing and/or further crossing of the deposit. Progeny also encompasses material that is obtained by vegetative propagation or another form of multiplication.

The invention further relates to the germplasm of plants of the invention. The germplasm is constituted by all inherited characteristics of an organism and according to the invention encompasses at least the trait of the invention. The germplasm can be used in a breeding program for the development of plants that exhibit the reduced leaf area phenotype. The use of germplasm that comprises the modified DCAF8 gene of the invention in breeding is also part of the present invention.

The invention also relates to the use of the modified DCAF8 gene of the invention for producing a plant that exhibits the reduced leaf area phenotype. The plant is preferably a plant that belongs to the Cucurbitaceae plant family, in particular a Cucumis sativus L. plant, most preferably a Cucumis sativus L. plant of the “slicer” and “long cucumber” cultivar groups.

Introduction of a modified DCAF8 gene of the invention can be done through introgression from a donor plant comprising said modified DCAF8 gene, in particular from another plant that shows reduced leaf area phenotype and in which the presence of a modified DCAF8 gene of the invention is identified, into a recipient plant that does not carry a modified DCAF8 gene, or which carries a modified DCAF8 gene heterozygously. Breeding methods such as crossing and selection, backcrossing, recombinant selection, or other breeding methods that result in the transfer of a genetic sequence from a donor plant to a recipient plant can be used. A donor plant, which is preferably a plant showing a reduced leaf area phenotype, can be of the same species or of a different and/or wild species. Difficulties in crossing between species can be overcome through techniques known in the art such as embryo rescue, or cis-genesis can be applied. A plant produced by such method is also a part of the invention.

The current invention also relates to the use of a plant of the invention as a crop, as a source of seed or as a source of propagation material.

The invention also relates to a marker sequence, in particular the marker sequence of SEQ ID No. 7, for the identification of the modified DCAF8 gene of the invention wherein the marker sequence detects a nucleotide substitution on position 592 of the wild type DCAF8 gene sequence of SEQ ID No. 2, or on a corresponding position of a homologous sequence that, in order of increased preference, has 70%, 75%, 80%, 85%, 90%, 91% 92%, 93%, 94%, 95%, 96%, 97%, 98%, 99% sequence identity to SEQ ID No. 2.

The use of a marker described herein for identification of the modified DCAF8 of the invention is also part of this invention.

The present invention relates to a method for identification of a plant comprising the modified DCAF8 gene of the invention, which plant can be identified phenotypically and/or genotypically, depending on whether it comprises the gene of the invention homozygously or heterozygously. A plant of the invention can be identified phenotypically, based on the fact that a plant comprising the modified DCAF8 gene homozygously exhibits a reduced leaf area phenotype. The genotypic identification of a plant of the invention comprises determining the presence of a modification in the DCAF8 gene, or in a homologous sequence thereof. Such genotypic identification can be followed by a phenotypic identification; i.e. analysing if the plant comprising the modification exhibits reduced leaf area phenotype.

Determining the presence of a modification in the DCAF8 gene of the invention comprises identification of any modification in SEQ ID No. 1 that leads to modification of protein function. Determining the presence of a modification includes determining the presence of any of the modifications as described herein. Determining the presence of a modification can be done through sequence comparison, which is known to the skilled person. Determining a modification is suitably done by using a marker that is designed to identify such modification as its sequence comprises that specific modification, in particular using a marker as described herein. Alternatively, determining the presence of a modification in the modified DCAF8 gene of the invention is done on the protein level and comprises the identification of any modification in SEQ ID No. 3, including any modification leading to a change in protein function.

The invention further relates to a method for selecting a plant that exhibits a reduced leaf area phenotype, comprising identifying the presence of a modification in the DCAF8 gene of the invention, and selecting a plant comprising a modification in the DCAF8 gene as a plant exhibiting reduced leaf area phenotype. Optionally, the method comprises a further step in which the leaf area is determined, for example by performing the leaf area measurement as described in Example 1. The selected plant obtained by the selection method is also a part of this invention.

The invention further relates to a method for seed production comprising growing a plant from a seed of the invention that comprises the modified DCAF8 gene of the invention, allowing the plant to produce a fruit with seed, harvesting the fruit, and extracting those seed. Production of the seed is suitably done by selfing or by crossing with another plant that is optionally also a plant of the invention or at least comprises the modified gene. Preferably, the plant grown from the seed produced as described herein exhibits reduced leaf area phenotype.

The invention also relates to a method for producing hybrid seed, comprising crossing a first parent plant with a second parent plant and harvesting the resultant hybrid seed, wherein the first parent plant and/or the second parent plant is a plant of the invention comprising the modified DCAF8 gene of the invention. Preferably, one of the parent plants comprises the modified DCAF8 gene of the invention homozygously.

The invention also relates to the hybrid seed produced by the method described herein and a hybrid plant grown from said hybrid seed. Preferably, the hybrid plant grown out of the hybrid seed comprises the modified gene of the invention heterozygously.

The present invention also relates to a method for producing a plant that comprises the gene of the invention, said method comprising the introduction of a modification in the DCAF8 gene.

The DCAF8 gene can be modified by different means known in the art, including mutagenesis. Mutagenesis comprises the random introduction of at least one modification to DNA by means of one or more chemical compounds, such as ethyl methanesulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast-neutron exposure, X-rays, gamma irradiation, and/or by insertion of genetic elements, such as transposons, T-DNA, retroviral elements. Mutagenesis also comprises the more specific, targeted introduction of at least one modification by means of homologous recombination, oligonucleotide-based mutation induction, zinc-finger nucleases (ZFNs), transcription activator-like effector nucleases (TALENs) or Clustered Regularly Interspaced Short Palindromic Repeat (CRISPR) systems.

The modified DCAF8 gene may be an exogenous DCAF8 gene introduced into a plant by a transgenic method or a cisgenic method. Use of a modified DCAF8 gene of the invention for developing a plant that exhibits a reduced leaf area phenotype, i.e. comprises smaller leaf area, comprises the introduction of a modified exogenous DCAF8 gene by a transgenic or a cisgenic method.

The modified DCAF8 gene may be part of a gene construct, which gene construct comprises a selectable marker, a promoter sequence, a DCAF8 gene sequence, and a terminator sequence.

The present invention is widely applicable to all plant species that have a functional orthologue of the DCAF8 gene in their genome, i.e. an orthologue that performs the same or a similar biological function. Identification of DCAF8 orthologues, i.e. DCAF8 genes in other species, can be performed in many crops, methods of which are known in the art. The present invention can for instance be applied to a plant belonging to a species selected from the group consisting of Cucumis sativus L., Cucumis melo L., Cucurbita pepo L., Cucurbita maxima (Duchesne) and Citrullus lanatus (Thunb.) Matsum. & Nakai.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Analysis of Leaf Area Phenotype

Plants producing a reduced leaf area phenotype can be recognized based on their smaller leaves. The average leaf area of a plant exhibiting reduced leaf area phenotype is at least 10% smaller than the average leaf area of a plant exhibiting normal growth phenotype (Table 1). The leaf area was measured in a non-destructive manner using LI-3100C-type digital leaf area meter, which uses a pixel counting method to provide the leaf area in cm². Leaf area measurements were done on two fully-grown intact leaves of individual plants carrying the modified DCAF8 gene of the invention, taken from the same height (the 16^{th} and 19^{th} leaves, or the 12^{th} and 18^{th} leaves), measured at the same time.

### Table 1

Leaf areas of 30 individual cucumber plants, selected for inbreeding. The leaf area was measured by digital leaf area meter and expressed in cm². Leaf area 1 (LA1) and 2 (LA2) are the areas of the 16^{th} and 19^{th} leaves, respectively. The average leaf area is calculated from LA1 and LA2 and rounded to the nearest integer.

| Plant ID | LA1 | LA2 | Average LA |
|---|---|---|---|
| 597-271 | 833 | 683 | 758 |
| 595-152 | 804 | 805 | 805 |
| 597-82 | 934 | 878 | 906 |
| 595-5 | 953 | 903 | 928 |
| 597-234 | 966 | 928 | 947 |
| 598-74 | 977 | 1102 | 1040 |
| 595-22 | 1036 | 1043 | 1040 |
| 598-2 | 1019 | 1068 | 1044 |
| 595-177 | 1030 | 1066 | 1048 |
| 598-123 | 1111 | 1079 | 1095 |
| 597-349 | 1068 | 1152 | 1110 |
| 598-118 | 1124 | 1256 | 1190 |
| 596-389 | 827 | 787 | 807 |
| 596-2 | 887 | 798 | 843 |
| 596-391 | 872 | 839 | 856 |
| 597-313 | 911 | 936 | 924 |
| 597-376 | 976 | 917 | 947 |
| 597-5 | 941 | 961 | 951 |
| 597-172 | 964 | 972 | 968 |
| 597-116 | 971 | 1059 | 1015 |
| 597-202 | 1023 | 1053 | 1038 |
| 597-41 | 817 | 755 | 786 |
| 595-91 | 824 | 807 | 816 |
| 597-330 | 837 | 838 | 838 |
| 597-390 | 880 | 804 | 842 |
| 595-323 | 861 | 826 | 842 |
| 595-257 | 891 | 849 | 870 |
| 597-239 | 987 | 851 | 919 |
| 595-182 | 1013 | 854 | 934 |
| 595-120 | 927 | 942 | 935 |

### Table 2

Leaf area of cucumber lines belonging to different haplotypes. The leaf area was measured by digital leaf area meter and expressed in cm². The average leaf area is calculated from the leaf areas of the 12^{th} and 18^{th} leaves of individual plants. In this experiment, the average leaf areas of groups of 5 individual plants of identical genetic background were compared. The haplotype was determined by KASP assay, and scored by A, B and H, wherein score A means that the plant comprises the wild type DCAF8 gene homozygously, score B means that the plant comprises the modified DCAF8 gene homozygously, while score H means that the plant comprises the modified DCAF8 gene heterozygously.

| Plant ID | Haplotype | LA1 | LA2 | Average LA |
|---|---|---|---|---|
| 596-391 | B | 872 | 839 | 856 |
| 597-330 | B | 837 | 838 | 838 |
| 595-323 | B | 861 | 826 | 844 |
| 596-389 | B | 827 | 787 | 807 |
| 596-2 | B | 887 | 798 | 843 |
| 597-41 | B | 817 | 755 | 786 |
| 595-91 | B | 824 | 807 | 816 |
| 598-74 | A | 977 | 1102 | 1040 |
| 598-2 | A | 1019 | 1068 | 1044 |
| 597-5 | A | 941 | 961 | 951 |
| 597-390 | B | 880 | 804 | 842 |
| 597-239 | A | 987 | 851 | 919 |
| 595-257 | A | 891 | 849 | 870 |
| 595-182 | A | 1013 | 854 | 934 |
| 595-120 | A | 927 | 942 | 935 |
| 598-118 | A | 1124 | 1256 | 1190 |
| 598-123 | A | 1111 | 1079 | 1095 |
| 597-116 | H | 971 | 1059 | 1015 |
| 597-349 | H | 1068 | 1152 | 1110 |
| 597-271 | H | 833 | 683 | 758 |
| 597-234 | H | 966 | 928 | 947 |
| 595-22 | H | 1036 | 1043 | 1040 |
| 595-177 | H | 1030 | 1066 | 1048 |
| 597-376 | H | 976 | 919 | 947 |
| 597-202 | H | 1023 | 1053 | 1038 |
| 595-152 | H | 804 | 805 | 805 |
| 597-82 | H | 934 | 878 | 906 |
| 597-313 | H | 911 | 936 | 924 |
| 597-172 | H | 964 | 972 | 968 |
| Average A | A | | | 998 |
| Average B | B | | | 829 |
| Average H | H | | | 959 |
| KK5.643 | B | | | 862 |
| KK5.682 | A | | | 968 |

### Example 2

### Identification of the Candidate Gene

The trait of reduced leaf area phenotype originated from a C. hardwickii introgression, source GBN104 (GBN104×BF-11). An introgression line library was created, consisting of homozygous lines each containing marker defined fragments from GBN104 in the BF-11 cultivated cucumber genetic background. With the help of the introgression line library, a major quantitative trait locus (QTL) linked to the trait of reduced leaf area phenotype was identified. This QTL corresponded to a region of about 160 kb, localized on chromosome 7 of Cucumis sativus L.

For finemapping two new populations were developed by crossing a normal growth phenotype plant (fatherline; KK5.682) with a reduced leaf area phenotype plant (motherline; KK5.643), derived from the above introgression, and developed up until the F4 generation. Plants were grown in glasshouse, with the average conditions being 16 h day time at 20° C. and 8 h night time at 17° C. Two leaves per plant were phenotyped, and 11 markers were used for genotyping. From the 1469 plants that were screened, 30 were selected for inbreeding and further phenotyping. The segregating lines from the earlier trial were sown and selected for presence or absence of the region. The datasets of these populations allowed to pinpoint a QTL corresponding to a genomic region between cs_BF11_v1_chr7_1.051.047 and cs_BF11_v1_chr7_1.085.251, of approximately 34 kb in size.

Comparison of the sequence data of this region with whole genome sequence databases allowed the identification of the gene: DDB1- and CUL4-associated factor 8 (DCAF8). A missense mutation was identified in the DCAF8 gene, at position 592 of SEQ ID Nr. 2, in plants carrying the C. hardwickii introgression.

The possible effects of the missense mutation on the protein structure and function were predicted using various computational tools [such as: PredictProtein (Bernhofer et al., 2021, https://predictprotein.org/), InterproScan (https://www.ebi.ac.uk/interpro/), and Find Individual Motif Occurrences—(FIMO)—https://meme-suite.org/meme/doc/fimo.html]. The analyses made clear that the missense mutation leads to a change in the secondary structure of the DCAF8 protein, resulting in the loss of a DNA/RNA binding site.

### Example 3

### Identification of DCAF8 Gene in Other Species of the Cucurbitaceae Family

A Basic Local Alignment Search Tool (BLAST) program was used to compare the DCAF8 gene as identified in SEQ ID No: 2 and the protein sequence as identified in SEQ ID No: 3 against the nucleotide coding sequences and protein sequences of other plant species belonging to the Cucurbitaceae family. This resulted in the identification of candidate DCAF8 orthologous genes. Multiple sequence alignment, using the Multiple Sequence Comparison by Log-Expectation (MUSCLE) program, using the identified DCAF8 protein sequences confirmed the high sequence similarity of DCAF8 in the Cucurbitaceae family (FIG. 3).

The invention is further described by the following numbered paragraphs:

1. A modified DCAF8 gene, the wild type of which gene having a coding sequence according to SEQ ID No. 2, encoding the protein of SEQ ID No. 3, or having a coding sequence and a protein sequence that show at least 70% sequence identity to SEQ ID No. 2 and at least 70% sequence similarity to SEQ ID No. 3, respectively, and wherein the modified DCAF8 gene comprises one or more nucleotides replaced, inserted and/or deleted relative to the wild type, and wherein the modified DCAF8 gene when present homozygously, confers to a cucurbit plant a reduced leaf area phenotype as compared to a cucurbit plant not having the modified gene.

2. The modified DCAF8 gene of paragraph 1, wherein the modified DCAF8 gene comprises a single nucleotide substitution leading to a missense mutation, on position 592 in SEQ ID No. 2, or on a corresponding position of a homologous sequence having at least 70% sequence identity to SEQ ID No. 2.

3. The modified DCAF8 gene of paragraph 1, wherein the modified DCAF8 gene comprises a mutation that leads to the substitution Gln->Glu on position 198 of SEQ ID No. 3 or on a corresponding position of a homologous amino acid sequence having at least 70% sequence similarity to SEQ ID No. 3.

4. A plant comprising the modified DCAF8 gene of any one of paragraphs 1 to 3.

5. The plant of paragraph 4, comprising the modified DCAF8 gene homozygously, which results in a reduction in leaf area of at least 10% as compared to a plant that is isogenic but does not possess the DCAF8 gene.

6. The plant of paragraph 5, wherein the leaf area is the average leaf area of two fully-grown intact leaves of individual plants, and wherein the fully grown leaves are chosen from the 12^{th} to 16^{th} (leaf 1) and the 16^{th} to 19^{th} (leaf 2) leaves of each plant.

7. A plant of any one of paragraphs 4 to 6, wherein the plant is a cucurbit plant.

8. The plant of any one of paragraphs 4 to 7, wherein the plant is a Cucumis sativus plant.

9. The plant of any one of paragraphs 4 to 8, wherein the plant is an agronomically elite plant, in particular a hybrid variety or an inbred line.

10. A plant of paragraph 8 or 9, wherein the modified gene is as present in a cucumber plant representative seed of which plant is deposited under deposit number NCIMB 43528.

11. A seed capable of growing into the plant of any one of paragraphs 4 to 10.

12. A fruit harvested from the plant of any one of paragraphs 4 to 10, wherein the fruit comprises the modified gene of any one of paragraphs 1 to 3.

13. Propagation material suitable for producing the plant of any one of paragraphs 4 to 10, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the modified DCAF8 gene of any one of paragraphs 1 to 3.

14. Use of the modified DCAF8 gene of any one of paragraphs 1 to 3 for producing a plant exhibiting a reduced leaf area phenotype.

15. A marker for the identification of the modified DCAF8 gene of any one of paragraphs 1 to 3, wherein the marker detects a single nucleotide substitution from cytosine to guanine, on position 592 in SEQ ID No. 2, or wherein the marker detects said substitution on a corresponding position of a homologous sequence that has 70% sequence identity to SEQ ID No. 2.

16. Use of the marker of paragraph 15 for the identification of a plant exhibiting a reduced leaf area phenotype.

17. A method for selecting the plant exhibiting a reduced leaf area phenotype, comprising identifying the presence of a modification in the DCAF8 gene of any one of paragraphs 1 to 3, optionally testing the plant for reduced leaf area phenotype, and selecting a plant comprising the modified DCAF8 gene.

18. A method for producing a plant exhibiting a reduced leaf area phenotype as set forth in paragraph 5 or 6, comprising the step of introducing a mutation into the DCAF8 gene, such that a protein product of the mutated DCAF8 gene leads to a reduced leaf area phenotype in the plant.

19. The method of paragraph 18, wherein the mutation is as defined in any one of paragraphs 1 to 3.

20. A method for producing a plant exhibiting reduced leaf area phenotype comprising the steps of:
a) crossing a first parent plant comprising the modified DCAF8 gene of any one of paragraphs 1 to 3 with a second parent plant to obtain an F1 population;
b) optionally performing one or more rounds of selfing and/or crossing with a plant from the F1 population to obtain a further generation population;
c) selecting from the population a plant that comprises the modified DCAF8 gene homozygously, as a plant exhibiting reduced leaf area phenotype.

21. A method for producing hybrid seed, comprising the steps of crossing a first parent plant with a second parent plant, wherein one or both parent plants are homozygous for the modified DCAF8 gene of any one of paragraphs 1 to 3, and harvesting the hybrid seed.

22. The hybrid seed produced by the method of paragraph 21.

23. A plant grown from the hybrid seed of paragraph 22.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### Sequence Information

TABLE 1
Sequences

| ID No. | Description | Sequence |
|---|---|---|
| SEQ ID | Wild type | TACGTAGAAAACCATTGCTAGAACTGACGAAGAAA |
| No. 1 | genomic | AATATTTTTATCATTTAGAATTTTTCATGTTTCGAGT |
| | DNA | ATATCTCTATACCTCTCAAGAAAAAAAAAAGAGTA |
| | sequence of | AAAGTAAATCTATATAATGTAATTTTCCCCTCAAAA |
| | CsDCAF8 | GAACAATTATATACCCTAATGATGATACTGATTCCA |
| | | TAAGAACTTCAAAAAGAAAAAACAAAACGAACAG |
| | | AAGAATGCACAAACTGAATTTACAGCCAACGTGGT |
| | | AGGAATTCATGATAATAACTAAGAGATCTTTTAGCT |
| | | ATTAATTCAGATGAAAATTTGTAAGAAGATTGATA |
| | | ATGGCATGCCGGATTCCTATTTTATGCACAATTAGC |
| | | TACCATGTTATTCAAATTTAGAGCTAAGAGTCAGCA |
| | | TAAACCTTCTCAATCGATATCAATCATCTTCACCAC |
| | | TATCATCATTATAATCTGAAGAGTCATCGCTGTTGC |
| | | CATTGTTGAATGTCCTTATCAACTCTAGAAGTTCCC |
| | | TACTTACAGTAGGAGGTTCTCTTCCACTTTCAGGGC |
| | | TCGAAGTCTGTCTTCGCAGTGAAAACAGTTGCATCA |
| | | TCAGTTCCTCCGGTAAGGCTAAGCGGTGCATCCATC |
| | | CTCTGGGCTTTGGTTTCTGTTGCAGAGTTGAAAATA |
| | | ATTAGATCAGAAGAATGGTATGAACATTGAGTCAA |
| | | CTACTGACATCATCGAAAATTTCATCAAATCTATTA |
| | | GCACTATCACTGAAAGCCATATCAAAATTCTATTCA |
| | | GTTTTTAACATCACTGTCATATCAAGTTCCCCTTGTC |
| | | ATCATCCCCATCATCACCTTCGTCTTCTTTATAATAA |
| | | TCATTGTTGAAAACAATGCCAATCAAGATGAACCCT |
| | | ACGTTCTTCAACAAATGCAGGAACTGGACCATGTA |
| | | ATTTATAGACACCAAGGAAATGACCCTCAGCTCAT |
| | | ATCATCTACAGAGTGTAAAGAAAAAGGGTATTTTG |
| | | CCAGAGTCCTAATGACCTTTAAAGGTTAAGAACAG |
| | | TTGCCTAGGAAACTCTATGGAATCTGGATAACTGTT |
| | | GAGCTTATGAGGCTATCTCGATTGCCCAGAGGTTTC |
| | | TTTCATTTTATTGAACAAGAAAAGACTACCACATGC |
| | | CCGCATCATGTTCTTATGGCATTAATTAGTCATGGA |
| | | AATTTATTTAGAAAATACTTGGTATTCCTATTTGTTC |
| | | TAAAGAACCAACTGCTGTCTGATCTTTTTGAGAAAG |
| | | CTTATCATTTGTCAAATAAAGCAACTCAAATACTTA |
| | | TTAAAGACATCATAAAAGATTACTTCAATTGGTGAT |
| | | GTAGAATGACAAATTTATACGACATCGATCAAAAT |
| | | GCTCTTATTTTAGGAGAGTGCACAAGAGAAAGAGT |
| | | GTTCAAGTACCTCAAATTATACGGATTATCTTGTAG |
| | | TTATTCTATCTTAGACATTGCAATATCGATGGGGTT |
| | | CTAGCAGATTGTTTATTCTTATTTGTATGACTACAA |
| | | GTCTAACGAAATACAAGAGGAAAAAACAACAGAA |
| | | ACTCATCCGGAGAAGCAGAAAACAGCAACACACAG |
| | | ATTATCCGTTGTTAAGCTCAAAAATGAAAGGAAAA |
| | | TATCCCATGTGATTATACTTGGAAAAATACAGGGCA |
| | | CATGGATTGCCATTAACTCAATATGTAAAGATTGAT |
| | | TTTTTTTTTTTTGCTGATCATAATGATGATGATATAT |
| | | TGACGGCAATAGTAACACATGGTAGTAGTATGAAA |
| | | AGGCCAACAAATACCAGAGTTCAGAATGCGAAAAT |
| | | GCAAGTGCTATTATCCTTATGGCAAAGGATGTTTTA |
| | | TTCAATATACAGGATAGAAACCTCAGGAATGCAAA |
| | | GAGTCCAATGCCATTCAATAAGCTGAAGTAAAGCA |
| | | TTAGTATTGGTATTAAACTGAAGAAGAAAGATGAT |
| | | ATTCCTACCTGTTCAATTTTCTCAGGAAGAGTAGCT |
| | | CTTTCATGGGCCTTTGGAGTCCATATCTTGACATCA |
| | | CTTTCAATTCCACTACTAGCAAGCATAGTCATATGA |
| | | GGATGGGGCTCGATGCAGTTCACGACATCCTCGTCA |
| | | GCTTCCATGACACGGATTAGATCTCCACCTTTTTTC |
| | | CTCCATATAAATATTCTGCCACAATCAGAACCACTA |
| | | ACGACATACTCACATTTAGGTCCAAAAAAATTGAC |
| | | CCCTTTAACTGTCTCACAGTTTCTATGTCCTTTATAA |
| | | ACTTGTGGAATACCATTGTCAACGTTTTCAGCTCCC |
| | | ATTTCACTTGCATCACTACTCAGAGATAGTGGAGGG |
| | | ACTGGATTTGGGTTTGGGCCCAAACCCATATCCCTA |
| | | TTAAAGAGATAAATGGATTCATCATTGTATGAGAC |
| | | AAGGAGCTCACTGAGCTCTGAGAAGGCTAGACCTG |
| | | TTATTCCCACTTGCTCATCACCAATCAAGTGAGGGG |
| | | GGCAATAGCAGTCTGCCAGTTGACCAAAATCAGTG |
| | | GAACCATCCTCCCCAGACTTGCGAATATCATACAAT |
| | | CGTGCATACTCATCTGAACCAGCAACAACAAAGAG |
| | | ATTTGGATTTCTGGGATCAATCACAATTGCATTAAG |
| | | CTCAATCGATGACATGTAACCTGCCCTATTGTCAAC |
| | | TGATTGGCAAGTGAACAGCTCAACGGCATCCCCAG |
| | | TTCTTAGATCAAACTATAAAATTAGTAAAGTCAGTA |
| | | TCACATGCAATTTAGCAATAATAAAAACGAAGGGT |
| | | CATTGGAAAACGTCTTTATTTCATGCCATAAGTTCA |
| | | GTAATAAATTTGCAAAACATGTGAACTTCTTCCGCT |
| | | GCGGAAAAGTATTTAACTTATCAAGTTGCAATAATA |
| | | CTTGGAATGTTGGTGGAACATAATATAACAGCAGA |
| | | AAGAGCAAATCGAAACTTACACGTTGCACCAATCC |
| | | ATCTTCACCACAAGTATAGAACATATAAGGACTAC |
| | | CAGGTTCAATGGCCAACTTATGAGCTCGCCCTAGAT |
| | | GATTGCCGAGTAGTGAAGTTTCCACATGCCCACTAT |
| | | TCAGAATCTGAGCATGCCTCACCTGAATTTCAGCAA |
| | | ATAATACTTTTTAAGAAATGAATGTTTAGTCTAGAG |
| | | ACAAATAGAAAAGCTTTACAGCGAGAAAAAACGAA |
| | | GTAAAACTTCTGCTGACAGAATACACAAGCAGTTT |
| | | AACTTAAGTTCAGAAAACAACATAATTCTAGTCGG |
| | | AAGAGGCATAAGTGGATTTCGAAAATATTAATGGT |
| | | ATATTGCAATTCTAGCGATCACTTTCAATGTATCTTT |
| | | TCCATGGGTAACAAAAAAATTGTGGCCCAGGCCAA |
| | | CCAAATTTGTGAAAATTTTAAAATACCTGGACACAC |
| | | AGGCCTGAAACATACATTTCAAGGATATCGGTGAA |
| | | TTAGGTATATCATTGACGTATCAGCATGTATTTGAG |
| | | ATGCATTTTAATAGCAAACCATTATGTATCAGCATT |
| | | CATTTAATGTGCATTTCAGGCATAACATTGGTGTAT |
| | | CAATATTATATAATTCCAACAGGAAAATAAATAAG |
| | | AATAACAATGGTGTATACAAAAGAGAAAATTTTAT |
| | | TGATCAATAGTTGTGAGTACAAGACAAGTACTTTAC |
| | | TTATAGCAAAAGTGCACTAATACTAATCCTAATTAA |
| | | TAAAAGAAACTTAATCCTGATCCTAAACAATAAGA |
| | | GAAACTATGGAATGCGTGGTTTGGCAGAAGATGGG |
| | | GGCAAGAAAGCAAATATATGTTCCATTAGAAGCTA |
| | | AAAGGAGCTGGTTGCCATTTAGAGATGTTGGACCA |
| | | ACTTCAGTATTTCTTGGATTTCTCTTCATGTGGGTGG |
| | | CAAGTATAGCCTCTCTGCTGATGCTGGTGTTTTGAA |
| | | AGACTTTTTCGAGCTTCCTCTAAATTTTGGATTAAT |
| | | ATGCCAAGACAGCTACAAGGCAGCCGGTTACATCT |
| | | TCTTGCAAGTTTTTAGATTCAATTGGGTTCCTTATGA |
| | | GATAAATTTTTTGTTATCCTTTGTATTTAGATATCTA |
| | | TGGCTGTGGTTCTGTTTAGCTTGATTTTTTTTCTGTT |
| | | GGAATTCGTTTTTTTAGCTTGTCATCTCCTTTGTTGC |
| | | CTAAGTTTAGCTTTTGTTATTGATTGAATTCTTCTCT |
| | | TTTATCTTGCTCTTTGTATAACACTCTTGTACTTTGA |
| | | GCTTAAATCTCATTTATTAATAATAAATAGACTTGT |
| | | CTTCATTTCAAAAAAAAATAATAAGAGAAACTAAT |
| | | CCTAATCCTAATCCTAATCCTAATAAACCAAACAAA |
| | | CTAATCCTAATCCTAATAAATTAAGGATTTGACCAT |
| | | AATACCCTATTCCTACTACATCATTCTTATCCCTAA |
| | | AATAACTTGTCCTCAAGTTTTAAAACGAAAATGAA |
| | | GGGAAAAATAAAAAAAATAACCTAGTCGAATGGAG |
| | | ACATTGGGGGAACATCCAACATCAATAATATCCAA |
| | | TAAGGAGGGTTGTGGACATTTAAGGTTAATTAAAA |
| | | AAAAAAAGGAAAACCGGCGAAAGGATAGTGAAAA |
| | | CATCCACTTCATATTGCTCCTTCTTCTTCCTTTATTG |
| | | ATTGTTGTGTAAATTGTGGTTCTTCCAAGATTAGTG |
| | | TAGTCCAAAATTCATCTTCATCCTTGTAGCAAATTG |
| | | TTTCTTCATTTTTGTGTGCAAAAACATCAATTTTGAT |
| | | TGGTTCGGTTCCCTCTGCAACACTGCCAACTTCATC |
| | | TTCTTCGTCCAAGGTAGCAATAGCTCCCCGCTTTTT |
| | | GTTGTCATCGATTTTCACTTCGACGCTTAGAGTTAC |
| | | GATCGTATTCTCTTTCATGTTTGAAATTTTAGATGG |
| | | AATATTCCTTGATTTTTCCTCAATTTTCGAGGACGTT |
| | | TTCTCTTTCAAATTAGGTAGCTTATTTCCAATTTATG |
| | | GCCACAGAAAATTGATATTGTTTTCTAATTGATTGA |
| | | GTGGCAAGAGCATGCGTTCGGTTCGACTAAACAAA |
| | | AGATACCAGCAATAGAGCTTTTCATTGACCACAAGT |
| | | GTCGGGTTGATGTCTTCAGTGATAGGGCAGTTGTTT |
| | | GCTCCTCCTCTTCAAAGGTTGTCACGGATAAGGGTG |
| | | AGCATGATAGACCAAAAAATCGAGCCACCGAACAA |
| | | ACCGAAGTCTATCGGTCGGAGAATGAGAGGGGTTG |
| | | GTCGATGCCGGTTTGGGGAAGTTCCAAATCGAGAA |
| | | ATTTTTAAAAATATTTCAAAATATTAAACCGACAAA |
| | | CATTTACTCATCGACGATCGAGATCGGTTTGCACGT |
| | | TTACTAAACCAACCATAGTTGATTGGGTGGCGGTTT |
| | | GGTTGAAAACCAATTTCGACCAACCAATGATCACC |
| | | CCTAGTCAATCGGTCACTGCCGGTAGGCTTTCTTCT |
| | | CACTCGCCATCAGGTTCAAGTTTCTTGGAGTTTCGA |
| | | TACCGATTGGCTATTCCAAATGTGAGAATTTTCTTG |
| | | AATATCAAAAGTTGTAAATACAAGAAAACAAGTAC |
| | | TTTATTTATAGCAAAAGCAAACTAATCCTAATCCTA |
| | | ATCCTAATCCTAATCCTAATCCTAATCCTAATCCTA |
| | | ATTAATAAAAGAAACTTATCCCAATCCTAAATAATA |
| | | AAAGAGACTTAATCCTAATTCTAAAAAATAAAAGA |
| | | AACTAATCCTAATTCTAAAAAGTAAAAGAAACTAA |
| | | TCCTAATTCTAAATAATAAAAGAAACTAATCCTAAT |
| | | CCTATTAAACCGAAGAAACTCATCTTATTCCTAATC |
| | | CTAATAAATTAAGGATTTGACCATAATACCTATTCC |
| | | TACTACATCAAAAAAGCTATGACTATAACTTAATTT |
| | | GAGAGAAAGAACAACTTAATTATTAATTTAAACAA |
| | | ATCTTATAGTTTTATTAATTTATAAAAATAATTGTTG |
| | | ATATCGATATTTGAATATCTGTAAAATAGAAATCTC |
| | | ATTATCAATGTCAATGTCATGGATGGTTTTTGTGTTT |
| | | CATGTATCACACAACTAAGAAAAGTGAAAAATAAA |
| | | AATTCAAAGCAATTTGCTATAATTGTAATACTCAAA |
| | | AACCGTCCAAATACACCAATATACTTCGCTGTCCAT |
| | | TACAATTTTTCTTTTCTAAATTATAATTTAATAAATC |
| | | TATGTCAGTAAACAACATGACTATTGTTGGGAGAA |
| | | GAAAAGTAAATGTTCTGCTGACAACAAATGAACAG |
| | | AAAACTTTACTTTTAAAATAAAATAAATAAACAGC |
| | | AAACTTTACTTAAATTGACATAACTTCATAAAATTA |
| | | ATATTGGGCATCAAGAAGAAATGGTCAAGTTTGAT |
| | | GGATAATGGAGAGGCTAGAATTTTGGAACAGAAAA |
| | | AACCAAAAAAGCGATTCAAACGACAAATTCACCGG |
| | | AAAACCTCCTTTCAATCCCACTCCAGGTAATGTTAT |
| | | TTCGAAAGACTGTCTGTGAAAACGTTAGGAACCAA |
| | | GGTAGTATCGAATGCCTTTGTCCCACATTGGTTAAA |
| | | ATATGATGACCAATGTGGTACTTGAGTGGCTTGGCT |
| | | TTCCAATCCAATAGCTGGTTTTTGGGTGTGGTTCTC |
| | | CATGGTGCTTAAGAACCTAACAGAAAATGGCTTCA |
| | | TTTAGATCAAGAAAAATTCAACAAGGCAAGGTTTC |
| | | ATAAATTTTTATAACATCCAATGTAGTGGAAAGAG |
| | | AATGGGTTTTGTGGTTATAGAAGGAGAAAATTATTT |
| | | GGATTTGGAGAAGTTTTTTTCTTTTTTTTTTTTTTTCT |
| | | TTTTTGCTGAGTAGAAGATTTTTTGAGAGGTATAAA |
| | | AAAGAGCTAAAACATTACCAGGAAGGAAATATCTT |
| | | TTGAGCAAGCAGCCAGATGCAGTGAAAGACCACAA |
| | | TCTCCCTCTTGAATGAACAACAGCCAAATATGGAAC |
| | | TCTAGTGATAGGAAGGGAAGAGTTCTAGATAATTT |
| | | ACAAAGGCACGTGCAAAAGAACTTTTGGAAAGCCA |
| | | GTGCCTCATCAAAGGCCTCTCAATGAAGGATGGAC |
| | | AATATTAGGCTGTTCTAGGTTGTAAAACTGGAGAA |
| | | GTTTAGTTAAAAAGATGTGTAGAACAGATAAGAAA |
| | | AGACTAAACATTGTAGATAGAATTGAAAGACGAAA |
| | | TTGCTTCCTCTGTAACTGGCCTCATATGTTCCCCAA |
| | | AATGTAAAAGGATGAAGAGATTATGAATCACCTCT |
| | | TTGTTTGTTGTGTTTGGGGCTAGAAGCCTAGGTGAT |
| | | TATCCCTTTTGAAAACCTTTGGTGTAAAAGCTGTTG |
| | | TTTCACGAATGGCATTGCATATTGGCTTGACTCCTA |
| | | GATTAATCTCAGATGGTGACTCGTAACCAAAGTTAA |
| | | AGAATTGTGGAAAGGTGTAAGTGAAGCTCCTTTCC |
| | | GATCCCTTTGGCCAGAGAGAAACAATAGTATTTTTA |
| | | ATGATAGTTACATAAGCGTCAATGACTTCTTGCTAA |
| | | ATGAAAAAGCAAACCCATATATAAAGAATAAGAGT |
| | | TCTTTAACTTGAGTGCTAAAACAAGTCACAATGATT |
| | | GAACAAAATGATATACCTGCCCATCTGCAGCACAA |
| | | GTCACAATGCTTTGATCATCCGTGTAAGGCATGATT |
| | | TTTGCTTGAAAAACATTGTTATTGTGACCAGACTGA |
| | | AAAGAGAGCTTGACACGCCCGGTTTCCCAATTCCA |
| | | GAGAATCACCCGCCTATCATCAGAGCCTGAAACTA |
| | | AAATATCACCAGCAGCATTAAAGCTCACGGTGTTC |
| | | ACACAACCTTTGTGCCTTTCCAGTTTCTTGTAAAGA |
| | | TCAAGTCGGAGCACGAGATCCTGAAATTACAGTAA |
| | | CAACAATAACGATCAATCCAGCGCGTCAAATAATA |
| | | AAAAGAAACCATAGATTTTCTACTACAGAAACAAA |
| | | ATCCTATTTATAGTGCAACACAATTAGATTCATTCC |
| | | GCTTGGTTTATTCAGTTCTGATCAAACAACGGAGGA |
| | | CAACATTTAACAATCAGAACAACTCTACAGCCTAG |
| | | CAGCCAACAGGATCAATGTGCATACAAAAAAATAT |
| | | GATAAAAACAGAAATCGAGAGATATGATCAGAAAA |
| | | GGATTATCCGGTGAGCAATACCTCCAAAGCACCAA |
| | | GACGGTGGGCAAAATTCCTAGTGGAAAGCTCTCCA |
| | | ACCTCCCGCTTCCAGACATTGACCACTGCCTTATTG |
| | | GAATCGTCAATTCTAGCCCTCTTGTTCATTATTGAA |
| | | TCCAAAAAAGAAAAAGAAAAAGAAAAAGAAAAAG |
| | | AAAAAAGGTTCCAAAAAGAAAAAGAAGAAGAATC |
| | | TCAGTATGATCAGATAAAGAGTCGATCGAGGAAGA |
| | | AGAGAGCGTTGGGAATGAAGAAGCACGTCGGCATC |
| | | GCCTGAGAAGAACTGAAATCGCCCGTTCACCGCTTT |
| | | AAGTAGAAGCAAAAGAGCATCTCATCGGAATGGCT |
| | | CAGAGGATTATATATTGATCGGCCTATTTGAGAACT |
| | | ACACGTTTACGCACAAAAGAAATAAACACATCTCA |
| | | ATGCAAAA |
| SEQ ID | Wild type | ATGAACAAGAGGGCTAGAATTGACGATTCCAATAA |
| No. 2 | CDS of | GGCAGTGGTCAATGTCTGGAAGCGGGAGGTTGGAG |
| | CsDCAF8 | AGCTTTCCACTAGGAATTTTGCCCACCGTCTTGGTG |
| | | CTTTGGAGGATCTCGTGCTCCGACTTGATCTTTACA |
| | | AGAAACTGGAAAGGCACAAAGGTTGTGTGAACACC |
| | | GTGAGCTTTAATGCTGCTGGTGATATTTTAGTTTCA |
| | | GGCTCTGATGATAGGCGGGTGATTCTCTGGAATTGG |
| | | GAAACCGGGCGTGTCAAGCTCTCTTTTCAGTCTGGT |
| | | CACAATAACAATGTTTTTCAAGCAAAAATCATGCCT |
| | | TACACGGATGATCAAAGCATTGTGACTTGTGCTGCA |
| | | GATGGGCAGGTGAGGCATGCTCAGATTCTGAATAG |
| | | TGGGCATGTGGAAACTTCACTACTCGGCAATCATCT |
| | | AGGGCGAGCTCATAAGTTGGCCATTGAACCTGGTA |
| | | GTCCTTATATGTTCTATACTTGTGGTGAAGATGGAT |
| | | TGGTGCAACGTTTTGATCTAAGAACTGGGGATGCCG |
| | | TTGAGCTGTTCACTTGCCAATCAGTTGACAATAGGG |
| | | CAGGTTACATGTCATCGATTCAGCTTAATGCAATTG |
| | | TGATTGATCCCAGAAATCCAAATCTCTTTGTTGTTG |
| | | CTGGTTCAGATGAGTATGCACGATTGTATGATATTC |
| | | GCAAGTCTGGGGAGGATGGTTCCACTGATTTTGGTC |
| | | AACTGGCAGACTGCTATTGCCCCCCTCACTTGATTG |
| | | GTGATGAGCAAGTGGGAATAACAGGTCTAGCCTTC |
| | | TCAGAGCTCAGTGAGCTCCTTGTCTCATACAATGAT |
| | | GAATCCATTTATCTCTTTAATAGGGATATGGGTTTG |
| | | GGCCCAAACCCAAATCCAGTCCCTCCACTATCTCTG |
| | | AGTAGTGATGCAAGTGAAATGGGAGCTGAAAACGT |
| | | TGACAATGGTATTCCACAAGTTTATAAAGGACATA |
| | | GAAACTGTGAGACAGTTAAAGGGGTCAATTTTTTTG |
| | | GACCTAAATGTGAGTATGTCGTTAGTGGTTCTGATT |
| | | GTGGCAGAATATTTATATGGAGGAAAAAAGGTGGA |
| | | GATCTAATCCGTGTCATGGAAGCTGACGAGGATGT |
| | | CGTGAACTGCATCGAGCCCCATCCTCATATGACTAT |
| | | GCTTGCTAGTAGTGGAATTGAAAGTGATGTCAAGA |
| | | TATGGACTCCAAAGGCCCATGAAAGAGCTACTCTTC |
| | | CTGAGAAAATTGAACAGAAACCAAAGCCCAGAGGA |
| | | TGGATGCACCGCTTAGCCTTACCGGAGGAACTGAT |
| | | GATGCAACTGTTTTCACTGCGAAGACAGACTTCGAG |
| | | CCCTGAAAGTGGAAGAGAACCTCCTACTGTAAGTA |
| | | GGGAACTTCTAGAGTTGATAAGGACATTCAACAAT |
| | | GGCAACAGCGATGACTCTTCAGATTATAATGATGAT |
| | | AGTGGTGAAGATGATTGA |
| SEQ ID | Wild type | MNKRARIDDSNKAVVNVWKREVGELSTRNFAHRLG |
| No. 3 | protein | ALEDLVLRLDLYKKLERHKGCVNTVSFNAAGDILVSG |
| | sequence of | SDDRRVILWNWETGRVKLSFQSGHNNNVFQAKIMPY |
| | CsDCAF8 | TDDQSIVTCAADGQVRHAQILNSGHVETSLLGNHLGR |
| | | AHKLAIEPGSPYMFYTCGEDGLVQRFDLRTGDAVELF |
| | | TCQSVDNRAGYMSSIQLNAIVIDPRNPNLFVVAGSDE |
| | | YARLYDIRKSGEDGSTDFGQLADCYCPPHLIGDEQVGI |
| | | TGLAFSELSELLVSYNDESIYLFNRDMGLGPNPNPVPP |
| | | LSLSSDASEMGAENVDNGIPQVYKGHRNCETVKGVN |
| | | FFGPKCEYVVSGSDCGRIFIWRKKGGDLIRVMEADED |
| | | VVNCIEPHPHMTMLASSGIESDVKIWTPKAHERATLPE |
| | | KIEQKPKPRGWMHRLALPEELMMQLESLRRQTSSPES |
| | | GREPPTVSRELLELIRTFNNGNSDDSSDYNDDSGEDD |
| SEQ ID | Modified | TACGTAGAAAACCATTGCTAGAACTGACGAAGAAA |
| No. 4 | genomic | AATATTTTTATCATTTAGAATTTTTCATGTTTCGAGT |
| | DNA | ATATCTCTATACCTCTCAAGAAAAAAAAAAGAGTA |
| | sequence of | AAAGTAAATCTATATAATGTAATTTTCCCCTCAAAA |
| | CsDCAF8 | GAACAATTATATACCCTAATGATGATACTGATTCCA |
| | | TAAGAACTTCAAAAAGAAAAAACAAAACGAACAG |
| | | AAGAATGCACAAACTGAATTTACAGCCAACGTGGT |
| | | AGGAATTCATGATAATAACTAAGAGATCTTTTAGCT |
| | | ATTAATTCAGATGAAAATTTGTAAGAAGATTGATA |
| | | ATGGCATGCCGGATTCCTATTTTATGCACAATTAGC |
| | | TACCATGTTATTCAAATTTAGAGCTAAGAGTCAGCA |
| | | TAAACCTTCTCAATCGATATCAATCATCTTCACCAC |
| | | TATCATCATTATAATCTGAAGAGTCATCGCTGTTGC |
| | | CATTGTTGAATGTCCTTATCAACTCTAGAAGTTCCC |
| | | TACTTACAGTAGGAGGTTCTCTTCCACTTTCAGGGC |
| | | TCGAAGTCTGTCTTCGCAGTGAAAACAGTTGCATCA |
| | | TCAGTTCCTCCGGTAAGGCTAAGCGGTGCATCCATC |
| | | CTCTGGGCTTTGGTTTCTGTTGCAGAGTTGAAAATA |
| | | ATTAGATCAGAAGAATGGTATGAACATTGAGTCAA |
| | | CTACTGACATCATCGAAAATTTCATCAAATCTATTA |
| | | GCACTATCACTGAAAGCCATATCAAAATTCTATTCA |
| | | GTTTTTAACATCACTGTCATATCAAGTTCCCCTTGTC |
| | | ATCATCCCCATCATCACCTTCGTCTTCTTTATAATAA |
| | | TCATTGTTGAAAACAATGCCAATCAAGATGAACCCT |
| | | ACGTTCTTCAACAAATGCAGGAACTGGACCATGTA |
| | | ATTTATAGACACCAAGGAAATGACCCTCAGCTCAT |
| | | ATCATCTACAGAGTGTAAAGAAAAAGGGTATTTTG |
| | | CCAGAGTCCTAATGACCTTTAAAGGTTAAGAACAG |
| | | TTGCCTAGGAAACTCTATGGAATCTGGATAACTGTT |
| | | GAGCTTATGAGGCTATCTCGATTGCCCAGAGGTTTC |
| | | TTTCATTTTATTGAACAAGAAAAGACTACCACATGC |
| | | CCGCATCATGTTCTTATGGCATTAATTAGTCATGGA |
| | | AATTTATTTAGAAAATACTTGGTATTCCTATTTGTTC |
| | | TAAAGAACCAACTGCTGTCTGATCTTTTTGAGAAAG |
| | | CTTATCATTTGTCAAATAAAGCAACTCAAATACTTA |
| | | TTAAAGACATCATAAAAGATTACTTCAATTGGTGAT |
| | | GTAGAATGACAAATTTATACGACATCGATCAAAAT |
| | | GCTCTTATTTTAGGAGAGTGCACAAGAGAAAGAGT |
| | | GTTCAAGTACCTCAAATTATACGGATTATCTTGTAG |
| | | TTATTCTATCTTAGACATTGCAATATCGATGGGGTT |
| | | CTAGCAGATTGTTTATTCTTATTTGTATGACTACAA |
| | | GTCTAACGAAATACAAGAGGAAAAAACAACAGAA |
| | | ACTCATCCGGAGAAGCAGAAAACAGCAACACACAG |
| | | ATTATCCGTTGTTAAGCTCAAAAATGAAAGGAAAA |
| | | TATCCCATGTGATTATACTTGGAAAAATACAGGGCA |
| | | CATGGATTGCCATTAACTCAATATGTAAAGATTGAT |
| | | TTTTTTTTTTTTGCTGATCATAATGATGATGATATAT |
| | | TGACGGCAATAGTAACACATGGTAGTAGTATGAAA |
| | | AGGCCAACAAATACCAGAGTTCAGAATGCGAAAAT |
| | | GCAAGTGCTATTATCCTTATGGCAAAGGATGTTTTA |
| | | TTCAATATACAGGATAGAAACCTCAGGAATGCAAA |
| | | GAGTCCAATGCCATTCAATAAGCTGAAGTAAAGCA |
| | | TTAGTATTGGTATTAAACTGAAGAAGAAAGATGAT |
| | | ATTCCTACCTGTTCAATTTTCTCAGGAAGAGTAGCT |
| | | CTTTCATGGGCCTTTGGAGTCCATATCTTGACATCA |
| | | CTTTCAATTCCACTACTAGCAAGCATAGTCATATGA |
| | | GGATGGGGCTCGATGCAGTTCACGACATCCTCGTCA |
| | | GCTTCCATGACACGGATTAGATCTCCACCTTTTTTC |
| | | CTCCATATAAATATTCTGCCACAATCAGAACCACTA |
| | | ACGACATACTCACATTTAGGTCCAAAAAAATTGAC |
| | | CCCTTTAACTGTCTCACAGTTTCTATGTCCTTTATAA |
| | | ACTTGTGGAATACCATTGTCAACGTTTTCAGCTCCC |
| | | ATTTCACTTGCATCACTACTCAGAGATAGTGGAGGG |
| | | ACTGGATTTGGGTTTGGGCCCAAACCCATATCCCTA |
| | | TTAAAGAGATAAATGGATTCATCATTGTATGAGAC |
| | | AAGGAGCTCACTGAGCTCTGAGAAGGCTAGACCTG |
| | | TTATTCCCACTTGCTCATCACCAATCAAGTGAGGGG |
| | | GGCAATAGCAGTCTGCCAGTTGACCAAAATCAGTG |
| | | GAACCATCCTCCCCAGACTTGCGAATATCATACAAT |
| | | CGTGCATACTCATCTGAACCAGCAACAACAAAGAG |
| | | ATTTGGATTTCTGGGATCAATCACAATTGCATTAAG |
| | | CT&AATCGATGACATGTAACCTGCCCTATTGTCAAC |
| | | TGATTGGCAAGTGAACAGCTCAACGGCATCCCCAG |
| | | TTCTTAGATCAAACTATAAAATTAGTAAAGTCAGTA |
| | | TCACATGCAATTTAGCAATAATAAAAACGAAGGGT |
| | | CATTGGAAAACGTCTTTATTTCATGCCATAAGTTCA |
| | | GTAATAAATTTGCAAAACATGTGAACTTCTTCCGCT |
| | | GCGGAAAAGTATTTAACTTATCAAGTTGCAATAATA |
| | | CTTGGAATGTTGGTGGAACATAATATAACAGCAGA |
| | | AAGAGCAAATCGAAACTTACACGTTGCACCAATCC |
| | | ATCTTCACCACAAGTATAGAACATATAAGGACTAC |
| | | CAGGTTCAATGGCCAACTTATGAGCTCGCCCTAGAT |
| | | GATTGCCGAGTAGTGAAGTTTCCACATGCCCACTAT |
| | | TCAGAATCTGAGCATGCCTCACCTGAATTTCAGCAA |
| | | ATAATACTTTTTAAGAAATGAATGTTTAGTCTAGAG |
| | | ACAAATAGAAAAGCTTTACAGCGAGAAAAAACGAA |
| | | GTAAAACTTCTGCTGACAGAATACACAAGCAGTTT |
| | | AACTTAAGTTCAGAAAACAACATAATTCTAGTCGG |
| | | AAGAGGCATAAGTGGATTTCGAAAATATTAATGGT |
| | | ATATTGCAATTCTAGCGATCACTTTCAATGTATCTTT |
| | | TCCATGGGTAACAAAAAAATTGTGGCCCAGGCCAA |
| | | CCAAATTTGTGAAAATTTTAAAATACCTGGACACAC |
| | | AGGCCTGAAACATACATTTCAAGGATATCGGTGAA |
| | | TTAGGTATATCATTGACGTATCAGCATGTATTTGAG |
| | | ATGCATTTTAATAGCAAACCATTATGTATCAGCATT |
| | | CATTTAATGTGCATTTCAGGCATAACATTGGTGTAT |
| | | CAATATTATATAATTCCAACAGGAAAATAAATAAG |
| | | AATAACAATGGTGTATACAAAAGAGAAAATTTTAT |
| | | TGATCAATAGTTGTGAGTACAAGACAAGTACTTTAC |
| | | TTATAGCAAAAGTGCACTAATACTAATCCTAATTAA |
| | | TAAAAGAAACTTAATCCTGATCCTAAACAATAAGA |
| | | GAAACTATGGAATGCGTGGTTTGGCAGAAGATGGG |
| | | GGCAAGAAAGCAAATATATGTTCCATTAGAAGCTA |
| | | AAAGGAGCTGGTTGCCATTTAGAGATGTTGGACCA |
| | | ACTTCAGTATTTCTTGGATTTCTCTTCATGTGGGTGG |
| | | CAAGTATAGCCTCTCTGCTGATGCTGGTGTTTTGAA |
| | | AGACTTTTTCGAGCTTCCTCTAAATTTTGGATTAAT |
| | | ATGCCAAGACAGCTACAAGGCAGCCGGTTACATCT |
| | | TCTTGCAAGTTTTTAGATTCAATTGGGTTCCTTATGA |
| | | GATAAATTTTTTGTTATCCTTTGTATTTAGATATCTA |
| | | TGGCTGTGGTTCTGTTTAGCTTGATTTTTTTTCTGTT |
| | | GGAATTCGTTTTTTTAGCTTGTCATCTCCTTTGTTGC |
| | | CTAAGTTTAGCTTTTGTTATTGATTGAATTCTTCTCT |
| | | TTTATCTTGCTCTTTGTATAACACTCTTGTACTTTGA |
| | | GCTTAAATCTCATTTATTAATAATAAATAGACTTGT |
| | | CTTCATTTCAAAAAAAAATAATAAGAGAAACTAAT |
| | | CCTAATCCTAATCCTAATCCTAATAAACCAAACAAA |
| | | CTAATCCTAATCCTAATAAATTAAGGATTTGACCAT |
| | | AATACCCTATTCCTACTACATCATTCTTATCCCTAA |
| | | AATAACTTGTCCTCAAGTTTTAAAACGAAAATGAA |
| | | GGGAAAAATAAAAAAAATAACCTAGTCGAATGGAG |
| | | ACATTGGGGGAACATCCAACATCAATAATATCCAA |
| | | TAAGGAGGGTTGTGGACATTTAAGGTTAATTAAAA |
| | | AAAAAAAGGAAAACCGGCGAAAGGATAGTGAAAA |
| | | CATCCACTTCATATTGCTCCTTCTTCTTCCTTTATTG |
| | | ATTGTTGTGTAAATTGTGGTTCTTCCAAGATTAGTG |
| | | TAGTCCAAAATTCATCTTCATCCTTGTAGCAAATTG |
| | | TTTCTTCATTTTTGTGTGCAAAAACATCAATTTTGAT |
| | | TGGTTCGGTTCCCTCTGCAACACTGCCAACTTCATC |
| | | TTCTTCGTCCAAGGTAGCAATAGCTCCCCGCTTTTT |
| | | GTTGTCATCGATTTTCACTTCGACGCTTAGAGTTAC |
| | | GATCGTATTCTCTTTCATGTTTGAAATTTTAGATGG |
| | | AATATTCCTTGATTTTTCCTCAATTTTCGAGGACGTT |
| | | TTCTCTTTCAAATTAGGTAGCTTATTTCCAATTTATG |
| | | GCCACAGAAAATTGATATTGTTTTCTAATTGATTGA |
| | | GTGGCAAGAGCATGCGTTCGGTTCGACTAAACAAA |
| | | AGATACCAGCAATAGAGCTTTTCATTGACCACAAGT |
| | | GTCGGGTTGATGTCTTCAGTGATAGGGCAGTTGTTT |
| | | GCTCCTCCTCTTCAAAGGTTGTCACGGATAAGGGTG |
| | | AGCATGATAGACCAAAAAATCGAGCCACCGAACAA |
| | | ACCGAAGTCTATCGGTCGGAGAATGAGAGGGGTTG |
| | | GTCGATGCCGGTTTGGGGAAGTTCCAAATCGAGAA |
| | | ATTTTTAAAAATATTTCAAAATATTAAACCGACAAA |
| | | CATTTACTCATCGACGATCGAGATCGGTTTGCACGT |
| | | TTACTAAACCAACCATAGTTGATTGGGTGGCGGTTT |
| | | GGTTGAAAACCAATTTCGACCAACCAATGATCACC |
| | | CCTAGTCAATCGGTCACTGCCGGTAGGCTTTCTTCT |
| | | CACTCGCCATCAGGTTCAAGTTTCTTGGAGTTTCGA |
| | | TACCGATTGGCTATTCCAAATGTGAGAATTTTCTTG |
| | | AATATCAAAAGTTGTAAATACAAGAAAACAAGTAC |
| | | TTTATTTATAGCAAAAGCAAACTAATCCTAATCCTA |
| | | ATCCTAATCCTAATCCTAATCCTAATCCTAATCCTA |
| | | ATTAATAAAAGAAACTTATCCCAATCCTAAATAATA |
| | | AAAGAGACTTAATCCTAATTCTAAAAAATAAAAGA |
| | | AACTAATCCTAATTCTAAAAAGTAAAAGAAACTAA |
| | | TCCTAATTCTAAATAATAAAAGAAACTAATCCTAAT |
| | | CCTATTAAACCGAAGAAACTCATCTTATTCCTAATC |
| | | CTAATAAATTAAGGATTTGACCATAATACCTATTCC |
| | | TACTACATCAAAAAAGCTATGACTATAACTTAATTT |
| | | GAGAGAAAGAACAACTTAATTATTAATTTAAACAA |
| | | ATCTTATAGTTTTATTAATTTATAAAAATAATTGTTG |
| | | ATATCGATATTTGAATATCTGTAAAATAGAAATCTC |
| | | ATTATCAATGTCAATGTCATGGATGGTTTTTGTGTTT |
| | | CATGTATCACACAACTAAGAAAAGTGAAAAATAAA |
| | | AATTCAAAGCAATTTGCTATAATTGTAATACTCAAA |
| | | AACCGTCCAAATACACCAATATACTTCGCTGTCCAT |
| | | TACAATTTTTCTTTTCTAAATTATAATTTAATAAATC |
| | | TATGTCAGTAAACAACATGACTATTGTTGGGAGAA |
| | | GAAAAGTAAATGTTCTGCTGACAACAAATGAACAG |
| | | AAAACTTTACTTTTAAAATAAAATAAATAAACAGC |
| | | AAACTTTACTTAAATTGACATAACTTCATAAAATTA |
| | | ATATTGGGCATCAAGAAGAAATGGTCAAGTTTGAT |
| | | GGATAATGGAGAGGCTAGAATTTTGGAACAGAAAA |
| | | AACCAAAAAAGCGATTCAAACGACAAATTCACCGG |
| | | AAAACCTCCTTTCAATCCCACTCCAGGTAATGTTAT |
| | | TTCGAAAGACTGTCTGTGAAAACGTTAGGAACCAA |
| | | GGTAGTATCGAATGCCTTTGTCCCACATTGGTTAAA |
| | | ATATGATGACCAATGTGGTACTTGAGTGGCTTGGCT |
| | | TTCCAATCCAATAGCTGGTTTTTGGGTGTGGTTCTC |
| | | CATGGTGCTTAAGAACCTAACAGAAAATGGCTTCA |
| | | TTTAGATCAAGAAAAATTCAACAAGGCAAGGTTTC |
| | | ATAAATTTTTATAACATCCAATGTAGTGGAAAGAG |
| | | AATGGGTTTTGTGGTTATAGAAGGAGAAAATTATTT |
| | | GGATTTGGAGAAGTTTTTTTCTTTTTTTTTTTTTTTCT |
| | | TTTTTGCTGAGTAGAAGATTTTTTGAGAGGTATAAA |
| | | AAAGAGCTAAAACATTACCAGGAAGGAAATATCTT |
| | | TTGAGCAAGCAGCCAGATGCAGTGAAAGACCACAA |
| | | TCTCCCTCTTGAATGAACAACAGCCAAATATGGAAC |
| | | TCTAGTGATAGGAAGGGAAGAGTTCTAGATAATTT |
| | | ACAAAGGCACGTGCAAAAGAACTTTTGGAAAGCCA |
| | | GTGCCTCATCAAAGGCCTCTCAATGAAGGATGGAC |
| | | AATATTAGGCTGTTCTAGGTTGTAAAACTGGAGAA |
| | | GTTTAGTTAAAAAGATGTGTAGAACAGATAAGAAA |
| | | AGACTAAACATTGTAGATAGAATTGAAAGACGAAA |
| | | TTGCTTCCTCTGTAACTGGCCTCATATGTTCCCCAA |
| | | AATGTAAAAGGATGAAGAGATTATGAATCACCTCT |
| | | TTGTTTGTTGTGTTTGGGGCTAGAAGCCTAGGTGAT |
| | | TATCCCTTTTGAAAACCTTTGGTGTAAAAGCTGTTG |
| | | TTTCACGAATGGCATTGCATATTGGCTTGACTCCTA |
| | | GATTAATCTCAGATGGTGACTCGTAACCAAAGTTAA |
| | | AGAATTGTGGAAAGGTGTAAGTGAAGCTCCTTTCC |
| | | GATCCCTTTGGCCAGAGAGAAACAATAGTATTTTTA |
| | | ATGATAGTTACATAAGCGTCAATGACTTCTTGCTAA |
| | | ATGAAAAAGCAAACCCATATATAAAGAATAAGAGT |
| | | TCTTTAACTTGAGTGCTAAAACAAGTCACAATGATT |
| | | GAACAAAATGATATACCTGCCCATCTGCAGCACAA |
| | | GTCACAATGCTTTGATCATCCGTGTAAGGCATGATT |
| | | TTTGCTTGAAAAACATTGTTATTGTGACCAGACTGA |
| | | AAAGAGAGCTTGACACGCCCGGTTTCCCAATTCCA |
| | | GAGAATCACCCGCCTATCATCAGAGCCTGAAACTA |
| | | AAATATCACCAGCAGCATTAAAGCTCACGGTGTTC |
| | | ACACAACCTTTGTGCCTTTCCAGTTTCTTGTAAAGA |
| | | TCAAGTCGGAGCACGAGATCCTGAAATTACAGTAA |
| | | CAACAATAACGATCAATCCAGCGCGTCAAATAATA |
| | | AAAAGAAACCATAGATTTTCTACTACAGAAACAAA |
| | | ATCCTATTTATAGTGCAACACAATTAGATTCATTCC |
| | | GCTTGGTTTATTCAGTTCTGATCAAACAACGGAGGA |
| | | CAACATTTAACAATCAGAACAACTCTACAGCCTAG |
| | | CAGCCAACAGGATCAATGTGCATACAAAAAAATAT |
| | | GATAAAAACAGAAATCGAGAGATATGATCAGAAAA |
| | | GGATTATCCGGTGAGCAATACCTCCAAAGCACCAA |
| | | GACGGTGGGCAAAATTCCTAGTGGAAAGCTCTCCA |
| | | ACCTCCCGCTTCCAGACATTGACCACTGCCTTATTG |
| | | GAATCGTCAATTCTAGCCCTCTTGTTCATTATTGAA |
| | | TCCAAAAAAGAAAAAGAAAAAGAAAAAGAAAAAG |
| | | AAAAAAGGTTCCAAAAAGAAAAAGAAGAAGAATC |
| | | TCAGTATGATCAGATAAAGAGTCGATCGAGGAAGA |
| | | AGAGAGCGTTGGGAATGAAGAAGCACGTCGGCATC |
| | | GCCTGAGAAGAACTGAAATCGCCCGTTCACCGCTTT |
| | | AAGTAGAAGCAAAAGAGCATCTCATCGGAATGGCT |
| | | CAGAGGATTATATATTGATCGGCCTATTTGAGAACT |
| | | ACACGTTTACGCACAAAAGAAATAAACACATCTCA |
| | | ATGCAAAA |
| SEQ ID | Modified | ATGAACAAGAGGGCTAGAATTGACGATTCCAATAA |
| No. 5 | CDS of | GGCAGTGGTCAATGTCTGGAAGCGGGAGGTTGGAG |
| | CsDCAF8 | AGCTTTCCACTAGGAATTTTGCCCACCGTCTTGGTG |
| | | CTTTGGAGGATCTCGTGCTCCGACTTGATCTTTACA |
| | | AGAAACTGGAAAGGCACAAAGGTTGTGTGAACACC |
| | | GTGAGCTTTAATGCTGCTGGTGATATTTTAGTTTCA |
| | | GGCTCTGATGATAGGCGGGTGATTCTCTGGAATTGG |
| | | GAAACCGGGCGTGTCAAGCTCTCTTTTCAGTCTGGT |
| | | CACAATAACAATGTTTTTCAAGCAAAAATCATGCCT |
| | | TACACGGATGATCAAAGCATTGTGACTTGTGCTGCA |
| | | GATGGGCAGGTGAGGCATGCTCAGATTCTGAATAG |
| | | TGGGCATGTGGAAACTTCACTACTCGGCAATCATCT |
| | | AGGGCGAGCTCATAAGTTGGCCATTGAACCTGGTA |
| | | GTCCTTATATGTTCTATACTTGTGGTGAAGATGGAT |
| | | TGGTGCAACGTTTTGATCTAAGAACTGGGGATGCCG |
| | | TTGAGCTGTTCACTTGCCAATCAGTTGACAATAGGG |
| | | CAGGTTACATGTCATCGATTgAGCTTAATGCAATTG |
| | | TGATTGATCCCAGAAATCCAAATCTCTTTGTTGTTG |
| | | CTGGTTCAGATGAGTATGCACGATTGTATGATATTC |
| | | GCAAGTCTGGGGAGGATGGTTCCACTGATTTTGGTC |
| | | AACTGGCAGACTGCTATTGCCCCCCTCACTTGATTG |
| | | GTGATGAGCAAGTGGGAATAACAGGTCTAGCCTTC |
| | | TCAGAGCTCAGTGAGCTCCTTGTCTCATACAATGAT |
| | | GAATCCATTTATCTCTTT |
| | | AATAGGGATATGGGTTTGGGCCCAAACCCAAATCC |
| | | AGTCCCTCCACTATCTCTGAGTAGTGATGCAAGTGA |
| | | AATGGGAGCTGAAAACGTTGACAATGGTATTCCAC |
| | | AAGTTTATAAAGGACATAGAAACTGTGAGACAGTT |
| | | AAAGGGGTCAATTTTTTTGGACCTAAATGTGAGTAT |
| | | GTCGTTAGTGGTTCTGATTGTGGCAGAATATTTATA |
| | | TGGAGGAAAAAAGGTGGAGATCTAATCCGTGTCAT |
| | | GGAAGCTGACGAGGATGTCGTGAACTGCATCGAGC |
| | | CCCATCCTCATATGACTATGCTTGCTAGTAGTGGAA |
| | | TTGAAAGTGATGTCAAGATATGGACTCCAAAGGCC |
| | | CATGAAAGAGCTACTCTTCCTGAGAAAATTGAACA |
| | | GAAACCAAAGCCCAGAGGATGGATGCACCGCTTAG |
| | | CCTTACCGGAGGAACTGATGATGCAACTGTTTTCAC |
| | | TGCGAAGACAGACTTCGAGCCCTGAAAGTGGAAGA |
| | | GAACCTCCTACTGTAAGTAGGGAACTTCTAGAGTTG |
| | | ATAAGGACATTCAACAATGGCAACAGCGATGACTC |
| | | TTCAGATTATAATGATGATAGTGGTGAAGATGATTG |
| | | A |
| SEQ ID | Modified | MNKRARIDDSNKAVVNVWKREVGELSTRNFAHRLG |
| No. 6 | protein | ALEDLVLRLDLYKKLERHKGCVNTVSFNAAGDILVSG |
| | sequence of | SDDRRVILWNWETGRVKLSFQSGHNNNVFQAKIMPY |
| | CsDCAF8 | TDDQSIVTCAADGQVRHAQILNSGHVETSLLGNHLGR |
| | | AHKLAIEPGSPYMFYTCGEDGLVQRFDLRTGDAVELF |
| | | TCQSVDNRAGYMSSIELNAIVIDPRNPNLFVVAGSDE |
| | | YARLYDIRKSGEDGSTDFGQLADCYCPPHLIGDEQVGI |
| | | TGLAFSELSELLVSYNDESIYLFNRDMGLGPNPNPVPP |
| | | LSLSSDASEMGAENVDNGIPQVYKGHRNCETVKGVN |
| | | FFGPKCEYVVSGSDCGRIFIWRKKGGDLIRVMEADED |
| | | VVNCIEPHPHMTMLASSGIESDVKIWTPKAHERATLPE |
| | | KIEQKPKPRGWMHRLALPEELMMQLFSLRRQTSSPES |
| | | GREPPTVSRELLELIRTFNNGNSDDSSDYNDDSGEDD |
| SEQ ID | Marker | YCCCCAGACTTGCGAATATCATACAATCGTGCATAC |
| No. 7 | sequence | TCATCTGAACCAGCAACAACAAAGAGATTTGGATT |
| | | TCTGGGATCAATCACAATTGCATTAAGCT[C/G]AAT |
| | | CGATGACATGTAACCTGCCCTATTGTCAACTGATTG |
| | | GCAAGTGAACAGCTCAACGGCATCCCCAGTTCTTA |
| | | GATCAAACTATAAAATTAGTAAAGTC |

1. An agronomically elite Cucurbitaceae plant comprising a modified DCAF8 gene, wherein:
(i) the wild type DCAF8 gene has a coding sequence comprising SEQ ID No. 2, and encodes a protein having a sequence comprising SEQ ID No. 3,
(ii) the modified DCAF8 gene has a coding sequence having at least 90% and up to 99% sequence identity to SEQ ID No. 2 and encodes a protein having at least 90% and up to 99% sequence identity to SEQ ID No. 3, and
(iii) the modified DCAF8 gene comprises a modification comprising one or more nucleotides of the wild type DCAF8 gene SEQ ID No. 2 replaced, inserted and/or deleted, and said modification comprising:
(a) a missense mutation at position 592 of SEQ ID No. 2; or,
(b) a missense mutation at a position corresponding to position 592 of SEQ ID No. 2 in a homologous coding sequence having at least 90% and up to 99% sequence identity to SEQ ID No. 2; or,
(c) a mutation detectable by genetic analysis using a marker having a sequence comprising SEQ ID No. 7; or,
(d) a mutation that leads to a Gln to Glu substitution at position 198 of SEQ ID No. 3; or,
(e) a mutation that leads to a Gln to Glu substitution at a position corresponding to position 198 of SEQ ID No. 3 in a homologous amino acid sequence having at least 90% and up to 99% sequence identity to SEQ ID No. 2; or,
(f) a mutation as is present in a cucumber plant, representative seed of which is deposited under Deposit Number NCIMB 43528; and
(iv) the modified DCAF8 gene, when homozygously present, confers to the Cucurbitaceae plant a reduced leaf area phenotype as compared to a Cucurbitaceae plant not having the modified gene.

2. The agronomically elite Cucurbitaceae plant of claim 1, wherein the modified DCAF8 gene comprises the missense mutation at position 592 of SEQ ID No. 2.

3. The agronomically elite Cucurbitaceae plant of claim 1, wherein the modified DCAF8 gene comprises the mutation that leads to the Gln to Glu substitution at position 198 of SEQ ID No. 3.

4. The agronomically elite Cucurbitaceae plant of claim 1, wherein the modified DCAF8 gene comprises the mutation detectable by genetic analysis using a marker having a sequence comprising SEQ ID No. 7.

5. The agronomically elite Cucurbitaceae plant of claim 1, comprising the modified DCAF8 gene homozygously, which results in a reduction in leaf area of at least 10% as compared to a Cucurbitaceae plant that is isogenic but does not possess the DCAF8 gene.

6. The agronomically elite Cucurbitaceae plant of claim 5, wherein the leaf area is an average leaf area of two fully-grown intact leaves of individual Cucurbitaceae plants, and wherein the fully grown leaves are chosen from the 12^{th} to 16^{th} (leaf 1) and the 16^{th} to 19^{th} (leaf 2) leaves of each plant.

7. The agronomically elite Cucurbitaceae plant of claim 1, wherein the agronomically elite Cucurbitaceae plant is an agronomically elite Cucumis sativus plant, and the modified DCAF8 gene comprises the missense mutation at position 592 of SEQ ID No. 2; or, the mutation detectable by genetic analysis using a marker having a sequence comprising SEQ ID No. 7; or, the mutation that leads to a Gln to Glu substitution at position 198 of SEQ ID No. 3; or, the mutation as is present in a cucumber plant, representative seed of which is deposited under Deposit Number NCIMB 43528.

8. The agronomically elite Cucumis sativus plant of claim 7, wherein the modified DCAF8 gene comprises the missense mutation at position 592 of SEQ ID No. 2.

9. The agronomically elite Cucumis sativus plant of claim 7, wherein the modified DCAF8 gene comprises the mutation that leads to a Gln to Glu substitution at position 198 of SEQ ID No. 3.

10. The agronomically elite Cucumis sativus plant of claim 7, wherein the modified DCAF8 gene is as present in a cucumber plant, representative seed of which plant is deposited under deposit number NCIMB 43528.

11. A seed of, or from, or that produces, the plant of claim 1, and comprises the modified DCAF8 gene.

12. A fruit of or from the agronomically elite Cucumis sativus plant of claim 7, comprising the modified DCAF8 gene.

13. Propagation material from or suitable for producing the plant of claim 1, wherein the propagation material is suitable for sexual reproduction, and comprises a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises the modified DCAF8 gene.

14. A seed or propagation material, wherein said seed or propagation material is of, or from, or produces, the agronomically elite Cucumis sativus plant of claim 7, and comprises the modified DCAF8 gene, and wherein the propagation material is suitable for sexual reproduction, and comprises a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem.

15. A method for identifying a Cucurbitaceae plant having a modified DCAF8 gene comprising genetic analysis of the plant using a marker, wherein the marker detects a single nucleotide substitution of cytosine to guanine at position 592 of SEQ ID No. 2, or wherein the marker detects said single nucleotide substitution at a corresponding position of a homologous coding sequence having at least 90% and up to 99% sequence identity to SEQ ID No. 2.

16. The method of claim 15 wherein the marker has the sequence of SEQ ID No: 7.

17. A method for selecting a Cucurbitaceae plant exhibiting a reduced leaf area phenotype, comprising:
a) identifying a Cucurbitaceae plant having a modified DCAF8 gene by genetic analysis of the Cucurbitaceae plant using a marker for identifying the modified DCAF8 gene, wherein the marker detects a single nucleotide substitution of cytosine to guanine at position 592 of SEQ ID No. 2, or wherein the marker detects said single nucleotide substitution at a corresponding position of a homologous coding sequence having at least 90% and up to 99% sequence identity to SEQ ID No. 2;
b) selecting the Cucurbitaceae plant that comprises the modified DCAF8 gene;
e) optionally further testing the selected Cucurbitaceae plant for the reduced leaf area phenotype; and
d) optionally selecting the further tested Cucurbitaceae plant that exhibits the reduced leaf area phenotype.

18. A method for producing a Cucurbitaceae plant exhibiting a reduced leaf area phenotype, comprising introducing a modification in a DCAF8 gene having a sequence comprising SEQ ID No: 2, or in another homologous sequence having at least 90% sequence identity to SEQ ID No: 2,
wherein the modified DCAF8 gene leads to a Gln to Glu substitution at position 198 of SEQ ID No. 3 or at a corresponding position of a homologous amino acid sequence having at least 90% and up to 99% sequence identity to SEQ ID No. 3.

19. A method for producing a Cucumis sativus plant exhibiting a reduced leaf area phenotype comprising introducing a modification in a DCAF8 gene having a sequence comprising SEQ ID No. 2, wherein the modified DCAF8 gene comprises a missense mutation at position 592 of SEQ ID No. 2; or, a mutation detectable by genetic analysis using a marker having a sequence comprising SEQ ID No. 7; or, a mutation that leads to a Gln to Glu substitution at position 198 of SEQ ID No. 3; or, a mutation as is present in a cucumber plant, representative seed of which is deposited under Deposit Number NCIMB 43528.

20. A method for producing a Cucurbitaceae plant exhibiting a reduced leaf area phenotype comprising the steps of:
a) crossing a first Cucurbitaceae parent with a second Cucurbitaceae parent plant that is isogenic to the first parent Cucurbitaceae plant to obtain an F1 population, wherein the first parent Cucurbitaceae plant is the Cucurbitaceae plant of claim 1;
b) optionally performing one or more rounds of selfing and/or crossing with a F1 Cucurbitaceae plant to obtain a further generation population;
c) selecting from the population resulting from the cross of step a), or from the further generation population of step b), a Cucurbitaceae plant that comprises the modified DCAF8 gene homozygously or exhibits the reduced leaf area phenotype.

21. A method for producing a hybrid Cucurbitaceae seed, comprising the steps of crossing a first Cucurbitaceae parent plant with a second Cucurbitaceae parent plant and harvesting the resultant hybrid Cucurbitaceae seed, wherein the first and second Cucurbitaceae parent plants are isogenic, and the first Cucurbitaceae parent plant and/or the second Cucurbitaceae parent plant is the Cucurbitaceae plant of claim 1 and comprises the modified DCAF8 gene homozygously.

22. The Cucurbitaceae hybrid seed produced by the method of claim 21.

23. A hybrid Cucurbitaceae plant grown from the hybrid Cucurbitaceae seed of claim 22.

24. The agronomically elite Cucumis sativus plant of claim 7 comprising the modified DCAF8 gene homozygously, which results in a reduction in leaf area of at least 10% as compared to a Cucumis sativus plant that is isogenic but does not possess the DCAF8 gene.

25. The agronomically elite Cucumis sativus plant of claim 24, wherein the leaf area is an average leaf area of two fully-grown intact leaves of individual Cucumis sativus plants, and wherein the fully grown leaves are chosen from the 12^{th} to 16^{th} (leaf 1) and the 16^{th} to 19^{th} (leaf 2) leaves of each plant.

26. The method of claim 17, wherein the Cucurbitaceae plant is a Cucumis sativus plant, and the method comprises:
a) identifying a Cucumis sativus plant having a modified DCAF8 gene by genetic analysis of the Cucurbitaceae plant using a marker for identifying the modified DCAF8 gene, wherein the marker detects a single nucleotide substitution from of cytosine to guanine at position 592 of SEQ ID No. 2;
b) selecting the Cucumis sativus plant that comprises the modified DCAF8 gene;
c) optionally further testing the selected Cucumis sativus plant for the reduced leaf area phenotype; and
d) optionally selecting the further tested Cucumis sativus plant that exhibits the reduced leaf area phenotype.

27. The method of claim 26 wherein the marker has a sequence comprising SEQ ID No. 7.

28. A method for producing a Cucumis sativus plant exhibiting a reduced leaf area phenotype comprising the steps of:
a) crossing a first Cucumis sativus parent plant with a second Cucumis sativus parent plant to obtain an F1 population, wherein the first Cucumis sativus parent plant is the Cucumis sativus plant of claim 7;
b) optionally performing one or more rounds of selfing and/or crossing with a F1 Cucumis sativus plant to obtain a further generation population;
c) selecting from the population resulting from the cross of step a), or from the further generation population of step b), a Cucumis sativus plant that comprises the modified DCAF8 gene homozygously or exhibits the reduced leaf area phenotype.

29. A method for producing a hybrid Cucumis sativus seed, comprising the steps of crossing a first Cucumis sativus parent plant with a second Cucumis sativus parent plant and harvesting the resultant hybrid Cucumis sativus seed, wherein the first Cucumis sativus parent plant and/or the second Cucumis sativus parent plant is the Cucurbitaceae plant of claim 7 and comprises the modified DCAF8 gene homozygously.

30. The Cucumis sativus hybrid seed produced by the method of claim 29.

31. An isolated nucleic acid molecule
(i) comprising a sequence having at least 90% and up to 99% sequence identity to SEQ ID No. 2 and encoding a protein having at least 90% and up to 99% sequence identity to SEQ ID No. 3, wherein:
(a) with reference to SEQ ID No. 2, the isolated nucleic acid molecule comprises a modification at position 592; or
(b) with reference to SEQ ID No. 3, the isolated nucleic acid molecule comprises a modification that leads to a Gln to Glu substitution at position 198 of SEQ ID No. 3; or,
(ii) comprising marker sequence SEQ ID No. 7.


SEQUENCE LISTING (8 sequences)

SEQ ID NO: 1 - DNA, 8280 bp, Cucumis sativus
  misc_feature 1..8280: note = Wild type genomic DNA sequence of CsDCAF8
  source 1..8280: mol_type = genomic DNA
tacgtagaaaaccattgctagaactgacgaagaaaaatatttttatcatttagaatttttcatgtttcgagtatatctctatacctctcaagaaaaaaaaaagagtaaaagtaaatctatataatgtaattttcccctcaaaagaacaattatataccctaatgatgatactgattccataagaacttcaaaaagaaaaaacaaaacgaacagaagaatgcacaaactgaatttacagccaacgtggtaggaattcatgataataactaagagatcttttagctattaattcagatgaaaatttgtaagaagattgataatggcatgccggattcctattttatgcacaattagctaccatgttattcaaatttagagctaagagtcagcataaaccttctcaatcgatatcaatcatcttcaccactatcatcattataatctgaagagtcatcgctgttgccattgttgaatgtccttatcaactctagaagttccctacttacagtaggaggttctcttccactttcagggctcgaagtctgtcttcgcagtgaaaacagttgcatcatcagttcctccggtaaggctaagcggtgcatccatcctctgggctttggtttctgttgcagagttgaaaataattagatcagaagaatggtatgaacattgagtcaactactgacatcatcgaaaatttcatcaaatctattagcactatcactgaaagccatatcaaaattctattcagtttttaacatcactgtcatatcaagttccccttgtcatcatccccatcatcaccttcgtcttctttataataatcattgttgaaaacaatgccaatcaagatgaaccctacgttcttcaacaaatgcaggaactggaccatgtaatttatagacaccaaggaaatgaccctcagctcatatcatctacagagtgtaaagaaaaagggtattttgccagagtcctaatgacctttaaaggttaagaacagttgcctaggaaactctatggaatctggataactgttgagcttatgaggctatctcgattgcccagaggtttctttcattttattgaacaagaaaagactaccacatgcccgcatcatgttcttatggcattaattagtcatggaaatttatttagaaaatacttggtattcctatttgttctaaagaaccaactgctgtctgatctttttgagaaagcttatcatttgtcaaataaagcaactcaaatacttattaaagacatcataaaagattacttcaattggtgatgtagaatgacaaatttatacgacatcgatcaaaatgctcttattttaggagagtgcacaagagaaagagtgttcaagtacctcaaattatacggattatcttgtagttattctatcttagacattgcaatatcgatggggttctagcagattgtttattcttatttgtatgactacaagtctaacgaaatacaagaggaaaaaacaacagaaactcatccggagaagcagaaaacagcaacacacagattatccgttgttaagctcaaaaatgaaaggaaaatatcccatgtgattatacttggaaaaatacagggcacatggattgccattaactcaatatgtaaagattgatttttttttttttgctgatcataatgatgatgatatattgacggcaatagtaacacatggtagtagtatgaaaaggccaacaaataccagagttcagaatgcgaaaatgcaagtgctattatccttatggcaaaggatgttttattcaatatacaggatagaaacctcaggaatgcaaagagtccaatgccattcaataagctgaagtaaagcattagtattggtattaaactgaagaagaaagatgatattcctacctgttcaattttctcaggaagagtagctctttcatgggcctttggagtccatatcttgacatcactttcaattccactactagcaagcatagtcatatgaggatggggctcgatgcagttcacgacatcctcgtcagcttccatgacacggattagatctccaccttttttcctccatataaatattctgccacaatcagaaccactaacgacatactcacatttaggtccaaaaaaattgacccctttaactgtctcacagtttctatgtcctttataaacttgtggaataccattgtcaacgttttcagctcccatttcacttgcatcactactcagagatagtggagggactggatttgggtttgggcccaaacccatatccctattaaagagataaatggattcatcattgtatgagacaaggagctcactgagctctgagaaggctagacctgttattcccacttgctcatcaccaatcaagtgaggggggcaatagcagtctgccagttgaccaaaatcagtggaaccatcctccccagacttgcgaatatcatacaatcgtgcatactcatctgaaccagcaacaacaaagagatttggatttctgggatcaatcacaattgcattaagctcaatcgatgacatgtaacctgccctattgtcaactgattggcaagtgaacagctcaacggcatccccagttcttagatcaaactataaaattagtaaagtcagtatcacatgcaatttagcaataataaaaacgaagggtcattggaaaacgtctttatttcatgccataagttcagtaataaatttgcaaaacatgtgaacttcttccgctgcggaaaagtatttaacttatcaagttgcaataatacttggaatgttggtggaacataatataacagcagaaagagcaaatcgaaacttacacgttgcaccaatccatcttcaccacaagtatagaacatataaggactaccaggttcaatggccaacttatgagctcgccctagatgattgccgagtagtgaagtttccacatgcccactattcagaatctgagcatgcctcacctgaatttcagcaaataatactttttaagaaatgaatgtttagtctagagacaaatagaaaagctttacagcgagaaaaaacgaagtaaaacttctgctgacagaatacacaagcagtttaacttaagttcagaaaacaacataattctagtcggaagaggcataagtggatttcgaaaatattaatggtatattgcaattctagcgatcactttcaatgtatcttttccatgggtaacaaaaaaattgtggcccaggccaaccaaatttgtgaaaattttaaaatacctggacacacaggcctgaaacatacatttcaaggatatcggtgaattaggtatatcattgacgtatcagcatgtatttgagatgcattttaatagcaaaccattatgtatcagcattcatttaatgtgcatttcaggcataacattggtgtatcaatattatataattccaacaggaaaataaataagaataacaatggtgtatacaaaagagaaaattttattgatcaatagttgtgagtacaagacaagtactttacttatagcaaaagtgcactaatactaatcctaattaataaaagaaacttaatcctgatcctaaacaataagagaaactatggaatgcgtggtttggcagaagatgggggcaagaaagcaaatatatgttccattagaagctaaaaggagctggttgccatttagagatgttggaccaacttcagtatttcttggatttctcttcatgtgggtggcaagtatagcctctctgctgatgctggtgttttgaaagactttttcgagcttcctctaaattttggattaatatgccaagacagctacaaggcagccggttacatcttcttgcaagtttttagattcaattgggttccttatgagataaattttttgttatcctttgtatttagatatctatggctgtggttctgtttagcttgattttttttctgttggaattcgtttttttagcttgtcatctcctttgttgcctaagtttagcttttgttattgattgaattcttctcttttatcttgctctttgtataacactcttgtactttgagcttaaatctcatttattaataataaatagacttgtcttcatttcaaaaaaaaataataagagaaactaatcctaatcctaatcctaatcctaataaaccaaacaaactaatcctaatcctaataaattaaggatttgaccataataccctattcctactacatcattcttatccctaaaataacttgtcctcaagttttaaaacgaaaatgaagggaaaaataaaaaaaataacctagtcgaatggagacattgggggaacatccaacatcaataatatccaataaggagggttgtggacatttaaggttaattaaaaaaaaaaaggaaaaccggcgaaaggatagtgaaaacatccacttcatattgctccttcttcttcctttattgattgttgtgtaaattgtggttcttccaagattagtgtagtccaaaattcatcttcatccttgtagcaaattgtttcttcatttttgtgtgcaaaaacatcaattttgattggttcggttccctctgcaacactgccaacttcatcttcttcgtccaaggtagcaatagctccccgctttttgttgtcatcgattttcacttcgacgcttagagttacgatcgtattctctttcatgtttgaaattttagatggaatattccttgatttttcctcaattttcgaggacgttttctctttcaaattaggtagcttatttccaatttatggccacagaaaattgatattgttttctaattgattgagtggcaagagcatgcgttcggttcgactaaacaaaagataccagcaatagagcttttcattgaccacaagtgtcgggttgatgtcttcagtgatagggcagttgtttgctcctcctcttcaaaggttgtcacggataagggtgagcatgatagaccaaaaaatcgagccaccgaacaaaccgaagtctatcggtcggagaatgagaggggttggtcgatgccggtttggggaagttccaaatcgagaaatttttaaaaatatttcaaaatattaaaccgacaaacatttactcatcgacgatcgagatcggtttgcacgtttactaaaccaaccatagttgattgggtggcggtttggttgaaaaccaatttcgaccaaccaatgatcacccctagtcaatcggtcactgccggtaggctttcttctcactcgccatcaggttcaagtttcttggagtttcgataccgattggctattccaaatgtgagaattttcttgaatatcaaaagttgtaaatacaagaaaacaagtactttatttatagcaaaagcaaactaatcctaatcctaatcctaatcctaatcctaatcctaatcctaatcctaattaataaaagaaacttatcccaatcctaaataataaaagagacttaatcctaattctaaaaaataaaagaaactaatcctaattctaaaaagtaaaagaaactaatcctaattctaaataataaaagaaactaatcctaatcctattaaaccgaagaaactcatcttattcctaatcctaataaattaaggatttgaccataatacctattcctactacatcaaaaaagctatgactataacttaatttgagagaaagaacaacttaattattaatttaaacaaatcttatagttttattaatttataaaaataattgttgatatcgatatttgaatatctgtaaaatagaaatctcattatcaatgtcaatgtcatggatggtttttgtgtttcatgtatcacacaactaagaaaagtgaaaaataaaaattcaaagcaatttgctataattgtaatactcaaaaaccgtccaaatacaccaatatacttcgctgtccattacaatttttcttttctaaattataatttaataaatctatgtcagtaaacaacatgactattgttgggagaagaaaagtaaatgttctgctgacaacaaatgaacagaaaactttacttttaaaataaaataaataaacagcaaactttacttaaattgacataacttcataaaattaatattgggcatcaagaagaaatggtcaagtttgatggataatggagaggctagaattttggaacagaaaaaaccaaaaaagcgattcaaacgacaaattcaccggaaaacctcctttcaatcccactccaggtaatgttatttcgaaagactgtctgtgaaaacgttaggaaccaaggtagtatcgaatgcctttgtcccacattggttaaaatatgatgaccaatgtggtacttgagtggcttggctttccaatccaatagctggtttttgggtgtggttctccatggtgcttaagaacctaacagaaaatggcttcatttagatcaagaaaaattcaacaaggcaaggtttcataaatttttataacatccaatgtagtggaaagagaatgggttttgtggttatagaaggagaaaattatttggatttggagaagtttttttctttttttttttttttcttttttgctgagtagaagattttttgagaggtataaaaaagagctaaaacattaccaggaaggaaatatcttttgagcaagcagccagatgcagtgaaagaccacaatctccctcttgaatgaacaacagccaaatatggaactctagtgataggaagggaagagttctagataatttacaaaggcacgtgcaaaagaacttttggaaagccagtgcctcatcaaaggcctctcaatgaaggatggacaatattaggctgttctaggttgtaaaactggagaagtttagttaaaaagatgtgtagaacagataagaaaagactaaacattgtagatagaattgaaagacgaaattgcttcctctgtaactggcctcatatgttccccaaaatgtaaaaggatgaagagattatgaatcacctctttgtttgttgtgtttggggctagaagcctaggtgattatcccttttgaaaacctttggtgtaaaagctgttgtttcacgaatggcattgcatattggcttgactcctagattaatctcagatggtgactcgtaaccaaagttaaagaattgtggaaaggtgtaagtgaagctcctttccgatccctttggccagagagaaacaatagtatttttaatgatagttacataagcgtcaatgacttcttgctaaatgaaaaagcaaacccatatataaagaataagagttctttaacttgagtgctaaaacaagtcacaatgattgaacaaaatgatatacctgcccatctgcagcacaagtcacaatgctttgatcatccgtgtaaggcatgatttttgcttgaaaaacattgttattgtgaccagactgaaaagagagcttgacacgcccggtttcccaattccagagaatcacccgcctatcatcagagcctgaaactaaaatatcaccagcagcattaaagctcacggtgttcacacaacctttgtgcctttccagtttcttgtaaagatcaagtcggagcacgagatcctgaaattacagtaacaacaataacgatcaatccagcgcgtcaaataataaaaagaaaccatagattttctactacagaaacaaaatcctatttatagtgcaacacaattagattcattccgcttggtttattcagttctgatcaaacaacggaggacaacatttaacaatcagaacaactctacagcctagcagccaacaggatcaatgtgcatacaaaaaaatatgataaaaacagaaatcgagagatatgatcagaaaaggattatccggtgagcaatacctccaaagcaccaagacggtgggcaaaattcctagtggaaagctctccaacctcccgcttccagacattgaccactgccttattggaatcgtcaattctagccctcttgttcattattgaatccaaaaaagaaaaagaaaaagaaaaagaaaaagaaaaaaggttccaaaaagaaaaagaagaagaatctcagtatgatcagataaagagtcgatcgaggaagaagagagcgttgggaatgaagaagcacgtcggcatcgcctgagaagaactgaaatcgcccgttcaccgctttaagtagaagcaaaagagcatctcatcggaatggctcagaggattatatattgatcggcctatttgagaactacacgtttacgcacaaaagaaataaacacatctcaatgcaaaa

SEQ ID NO: 2 - DNA, 1443 bp, Cucumis sativus
  misc_feature 1..1443: note = Wild type CDS of CsDCAF8
  source 1..1443: mol_type = genomic DNA
atgaacaagagggctagaattgacgattccaataaggcagtggtcaatgtctggaagcgggaggttggagagctttccactaggaattttgcccaccgtcttggtgctttggaggatctcgtgctccgacttgatctttacaagaaactggaaaggcacaaaggttgtgtgaacaccgtgagctttaatgctgctggtgatattttagtttcaggctctgatgataggcgggtgattctctggaattgggaaaccgggcgtgtcaagctctcttttcagtctggtcacaataacaatgtttttcaagcaaaaatcatgccttacacggatgatcaaagcattgtgacttgtgctgcagatgggcaggtgaggcatgctcagattctgaatagtgggcatgtggaaacttcactactcggcaatcatctagggcgagctcataagttggccattgaacctggtagtccttatatgttctatacttgtggtgaagatggattggtgcaacgttttgatctaagaactggggatgccgttgagctgttcacttgccaatcagttgacaatagggcaggttacatgtcatcgattcagcttaatgcaattgtgattgatcccagaaatccaaatctctttgttgttgctggttcagatgagtatgcacgattgtatgatattcgcaagtctggggaggatggttccactgattttggtcaactggcagactgctattgcccccctcacttgattggtgatgagcaagtgggaataacaggtctagccttctcagagctcagtgagctccttgtctcatacaatgatgaatccatttatctctttaatagggatatgggtttgggcccaaacccaaatccagtccctccactatctctgagtagtgatgcaagtgaaatgggagctgaaaacgttgacaatggtattccacaagtttataaaggacatagaaactgtgagacagttaaaggggtcaatttttttggacctaaatgtgagtatgtcgttagtggttctgattgtggcagaatatttatatggaggaaaaaaggtggagatctaatccgtgtcatggaagctgacgaggatgtcgtgaactgcatcgagccccatcctcatatgactatgcttgctagtagtggaattgaaagtgatgtcaagatatggactccaaaggcccatgaaagagctactcttcctgagaaaattgaacagaaaccaaagcccagaggatggatgcaccgcttagccttaccggaggaactgatgatgcaactgttttcactgcgaagacagacttcgagccctgaaagtggaagagaacctcctactgtaagtagggaacttctagagttgataaggacattcaacaatggcaacagcgatgactcttcagattataatgatgatagtggtgaagatgattga

SEQ ID NO: 3 - protein, 480 aa, Cucumis sativus
  REGION 1..480: note = Wild type protein sequence of CsDCAF8
MNKRARIDDSNKAVVNVWKREVGELSTRNFAHRLGALEDLVLRLDLYKKLERHKGCVNTVSFNAAGDILVSGSDDRRVILWNWETGRVKLSFQSGHNNNVFQAKIMPYTDDQSIVTCAADGQVRHAQILNSGHVETSLLGNHLGRAHKLAIEPGSPYMFYTCGEDGLVQRFDLRTGDAVELFTCQSVDNRAGYMSSIQLNAIVIDPRNPNLFVVAGSDEYARLYDIRKSGEDGSTDFGQLADCYCPPHLIGDEQVGITGLAFSELSELLVSYNDESIYLFNRDMGLGPNPNPVPPLSLSSDASEMGAENVDNGIPQVYKGHRNCETVKGVNFFGPKCEYVVSGSDCGRIFIWRKKGGDLIRVMEADEDVVNCIEPHPHMTMLASSGIESDVKIWTPKAHERATLPEKIEQKPKPRGWMHRLALPEELMMQLFSLRRQTSSPESGREPPTVSRELLELIRTFNNGNSDDSSDYNDDSGEDD

SEQ ID NO: 4 - DNA, 8280 bp, Cucumis sativus
  misc_feature 1..8280: note = Modified genomic DNA sequence of CsDCAF8
  source 1..8280: mol_type = genomic DNA
tacgtagaaaaccattgctagaactgacgaagaaaaatatttttatcatttagaatttttcatgtttcgagtatatctctatacctctcaagaaaaaaaaaagagtaaaagtaaatctatataatgtaattttcccctcaaaagaacaattatataccctaatgatgatactgattccataagaacttcaaaaagaaaaaacaaaacgaacagaagaatgcacaaactgaatttacagccaacgtggtaggaattcatgataataactaagagatcttttagctattaattcagatgaaaatttgtaagaagattgataatggcatgccggattcctattttatgcacaattagctaccatgttattcaaatttagagctaagagtcagcataaaccttctcaatcgatatcaatcatcttcaccactatcatcattataatctgaagagtcatcgctgttgccattgttgaatgtccttatcaactctagaagttccctacttacagtaggaggttctcttccactttcagggctcgaagtctgtcttcgcagtgaaaacagttgcatcatcagttcctccggtaaggctaagcggtgcatccatcctctgggctttggtttctgttgcagagttgaaaataattagatcagaagaatggtatgaacattgagtcaactactgacatcatcgaaaatttcatcaaatctattagcactatcactgaaagccatatcaaaattctattcagtttttaacatcactgtcatatcaagttccccttgtcatcatccccatcatcaccttcgtcttctttataataatcattgttgaaaacaatgccaatcaagatgaaccctacgttcttcaacaaatgcaggaactggaccatgtaatttatagacaccaaggaaatgaccctcagctcatatcatctacagagtgtaaagaaaaagggtattttgccagagtcctaatgacctttaaaggttaagaacagttgcctaggaaactctatggaatctggataactgttgagcttatgaggctatctcgattgcccagaggtttctttcattttattgaacaagaaaagactaccacatgcccgcatcatgttcttatggcattaattagtcatggaaatttatttagaaaatacttggtattcctatttgttctaaagaaccaactgctgtctgatctttttgagaaagcttatcatttgtcaaataaagcaactcaaatacttattaaagacatcataaaagattacttcaattggtgatgtagaatgacaaatttatacgacatcgatcaaaatgctcttattttaggagagtgcacaagagaaagagtgttcaagtacctcaaattatacggattatcttgtagttattctatcttagacattgcaatatcgatggggttctagcagattgtttattcttatttgtatgactacaagtctaacgaaatacaagaggaaaaaacaacagaaactcatccggagaagcagaaaacagcaacacacagattatccgttgttaagctcaaaaatgaaaggaaaatatcccatgtgattatacttggaaaaatacagggcacatggattgccattaactcaatatgtaaagattgatttttttttttttgctgatcataatgatgatgatatattgacggcaatagtaacacatggtagtagtatgaaaaggccaacaaataccagagttcagaatgcgaaaatgcaagtgctattatccttatggcaaaggatgttttattcaatatacaggatagaaacctcaggaatgcaaagagtccaatgccattcaataagctgaagtaaagcattagtattggtattaaactgaagaagaaagatgatattcctacctgttcaattttctcaggaagagtagctctttcatgggcctttggagtccatatcttgacatcactttcaattccactactagcaagcatagtcatatgaggatggggctcgatgcagttcacgacatcctcgtcagcttccatgacacggattagatctccaccttttttcctccatataaatattctgccacaatcagaaccactaacgacatactcacatttaggtccaaaaaaattgacccctttaactgtctcacagtttctatgtcctttataaacttgtggaataccattgtcaacgttttcagctcccatttcacttgcatcactactcagagatagtggagggactggatttgggtttgggcccaaacccatatccctattaaagagataaatggattcatcattgtatgagacaaggagctcactgagctctgagaaggctagacctgttattcccacttgctcatcaccaatcaagtgaggggggcaatagcagtctgccagttgaccaaaatcagtggaaccatcctccccagacttgcgaatatcatacaatcgtgcatactcatctgaaccagcaacaacaaagagatttggatttctgggatcaatcacaattgcattaagctgaatcgatgacatgtaacctgccctattgtcaactgattggcaagtgaacagctcaacggcatccccagttcttagatcaaactataaaattagtaaagtcagtatcacatgcaatttagcaataataaaaacgaagggtcattggaaaacgtctttatttcatgccataagttcagtaataaatttgcaaaacatgtgaacttcttccgctgcggaaaagtatttaacttatcaagttgcaataatacttggaatgttggtggaacataatataacagcagaaagagcaaatcgaaacttacacgttgcaccaatccatcttcaccacaagtatagaacatataaggactaccaggttcaatggccaacttatgagctcgccctagatgattgccgagtagtgaagtttccacatgcccactattcagaatctgagcatgcctcacctgaatttcagcaaataatactttttaagaaatgaatgtttagtctagagacaaatagaaaagctttacagcgagaaaaaacgaagtaaaacttctgctgacagaatacacaagcagtttaacttaagttcagaaaacaacataattctagtcggaagaggcataagtggatttcgaaaatattaatggtatattgcaattctagcgatcactttcaatgtatcttttccatgggtaacaaaaaaattgtggcccaggccaaccaaatttgtgaaaattttaaaatacctggacacacaggcctgaaacatacatttcaaggatatcggtgaattaggtatatcattgacgtatcagcatgtatttgagatgcattttaatagcaaaccattatgtatcagcattcatttaatgtgcatttcaggcataacattggtgtatcaatattatataattccaacaggaaaataaataagaataacaatggtgtatacaaaagagaaaattttattgatcaatagttgtgagtacaagacaagtactttacttatagcaaaagtgcactaatactaatcctaattaataaaagaaacttaatcctgatcctaaacaataagagaaactatggaatgcgtggtttggcagaagatgggggcaagaaagcaaatatatgttccattagaagctaaaaggagctggttgccatttagagatgttggaccaacttcagtatttcttggatttctcttcatgtgggtggcaagtatagcctctctgctgatgctggtgttttgaaagactttttcgagcttcctctaaattttggattaatatgccaagacagctacaaggcagccggttacatcttcttgcaagtttttagattcaattgggttccttatgagataaattttttgttatcctttgtatttagatatctatggctgtggttctgtttagcttgattttttttctgttggaattcgtttttttagcttgtcatctcctttgttgcctaagtttagcttttgttattgattgaattcttctcttttatcttgctctttgtataacactcttgtactttgagcttaaatctcatttattaataataaatagacttgtcttcatttcaaaaaaaaataataagagaaactaatcctaatcctaatcctaatcctaataaaccaaacaaactaatcctaatcctaataaattaaggatttgaccataataccctattcctactacatcattcttatccctaaaataacttgtcctcaagttttaaaacgaaaatgaagggaaaaataaaaaaaataacctagtcgaatggagacattgggggaacatccaacatcaataatatccaataaggagggttgtggacatttaaggttaattaaaaaaaaaaaggaaaaccggcgaaaggatagtgaaaacatccacttcatattgctccttcttcttcctttattgattgttgtgtaaattgtggttcttccaagattagtgtagtccaaaattcatcttcatccttgtagcaaattgtttcttcatttttgtgtgcaaaaacatcaattttgattggttcggttccctctgcaacactgccaacttcatcttcttcgtccaaggtagcaatagctccccgctttttgttgtcatcgattttcacttcgacgcttagagttacgatcgtattctctttcatgtttgaaattttagatggaatattccttgatttttcctcaattttcgaggacgttttctctttcaaattaggtagcttatttccaatttatggccacagaaaattgatattgttttctaattgattgagtggcaagagcatgcgttcggttcgactaaacaaaagataccagcaatagagcttttcattgaccacaagtgtcgggttgatgtcttcagtgatagggcagttgtttgctcctcctcttcaaaggttgtcacggataagggtgagcatgatagaccaaaaaatcgagccaccgaacaaaccgaagtctatcggtcggagaatgagaggggttggtcgatgccggtttggggaagttccaaatcgagaaatttttaaaaatatttcaaaatattaaaccgacaaacatttactcatcgacgatcgagatcggtttgcacgtttactaaaccaaccatagttgattgggtggcggtttggttgaaaaccaatttcgaccaaccaatgatcacccctagtcaatcggtcactgccggtaggctttcttctcactcgccatcaggttcaagtttcttggagtttcgataccgattggctattccaaatgtgagaattttcttgaatatcaaaagttgtaaatacaagaaaacaagtactttatttatagcaaaagcaaactaatcctaatcctaatcctaatcctaatcctaatcctaatcctaatcctaattaataaaagaaacttatcccaatcctaaataataaaagagacttaatcctaattctaaaaaataaaagaaactaatcctaattctaaaaagtaaaagaaactaatcctaattctaaataataaaagaaactaatcctaatcctattaaaccgaagaaactcatcttattcctaatcctaataaattaaggatttgaccataatacctattcctactacatcaaaaaagctatgactataacttaatttgagagaaagaacaacttaattattaatttaaacaaatcttatagttttattaatttataaaaataattgttgatatcgatatttgaatatctgtaaaatagaaatctcattatcaatgtcaatgtcatggatggtttttgtgtttcatgtatcacacaactaagaaaagtgaaaaataaaaattcaaagcaatttgctataattgtaatactcaaaaaccgtccaaatacaccaatatacttcgctgtccattacaatttttcttttctaaattataatttaataaatctatgtcagtaaacaacatgactattgttgggagaagaaaagtaaatgttctgctgacaacaaatgaacagaaaactttacttttaaaataaaataaataaacagcaaactttacttaaattgacataacttcataaaattaatattgggcatcaagaagaaatggtcaagtttgatggataatggagaggctagaattttggaacagaaaaaaccaaaaaagcgattcaaacgacaaattcaccggaaaacctcctttcaatcccactccaggtaatgttatttcgaaagactgtctgtgaaaacgttaggaaccaaggtagtatcgaatgcctttgtcccacattggttaaaatatgatgaccaatgtggtacttgagtggcttggctttccaatccaatagctggtttttgggtgtggttctccatggtgcttaagaacctaacagaaaatggcttcatttagatcaagaaaaattcaacaaggcaaggtttcataaatttttataacatccaatgtagtggaaagagaatgggttttgtggttatagaaggagaaaattatttggatttggagaagtttttttctttttttttttttttcttttttgctgagtagaagattttttgagaggtataaaaaagagctaaaacattaccaggaaggaaatatcttttgagcaagcagccagatgcagtgaaagaccacaatctccctcttgaatgaacaacagccaaatatggaactctagtgataggaagggaagagttctagataatttacaaaggcacgtgcaaaagaacttttggaaagccagtgcctcatcaaaggcctctcaatgaaggatggacaatattaggctgttctaggttgtaaaactggagaagtttagttaaaaagatgtgtagaacagataagaaaagactaaacattgtagatagaattgaaagacgaaattgcttcctctgtaactggcctcatatgttccccaaaatgtaaaaggatgaagagattatgaatcacctctttgtttgttgtgtttggggctagaagcctaggtgattatcccttttgaaaacctttggtgtaaaagctgttgtttcacgaatggcattgcatattggcttgactcctagattaatctcagatggtgactcgtaaccaaagttaaagaattgtggaaaggtgtaagtgaagctcctttccgatccctttggccagagagaaacaatagtatttttaatgatagttacataagcgtcaatgacttcttgctaaatgaaaaagcaaacccatatataaagaataagagttctttaacttgagtgctaaaacaagtcacaatgattgaacaaaatgatatacctgcccatctgcagcacaagtcacaatgctttgatcatccgtgtaaggcatgatttttgcttgaaaaacattgttattgtgaccagactgaaaagagagcttgacacgcccggtttcccaattccagagaatcacccgcctatcatcagagcctgaaactaaaatatcaccagcagcattaaagctcacggtgttcacacaacctttgtgcctttccagtttcttgtaaagatcaagtcggagcacgagatcctgaaattacagtaacaacaataacgatcaatccagcgcgtcaaataataaaaagaaaccatagattttctactacagaaacaaaatcctatttatagtgcaacacaattagattcattccgcttggtttattcagttctgatcaaacaacggaggacaacatttaacaatcagaacaactctacagcctagcagccaacaggatcaatgtgcatacaaaaaaatatgataaaaacagaaatcgagagatatgatcagaaaaggattatccggtgagcaatacctccaaagcaccaagacggtgggcaaaattcctagtggaaagctctccaacctcccgcttccagacattgaccactgccttattggaatcgtcaattctagccctcttgttcattattgaatccaaaaaagaaaaagaaaaagaaaaagaaaaagaaaaaaggttccaaaaagaaaaagaagaagaatctcagtatgatcagataaagagtcgatcgaggaagaagagagcgttgggaatgaagaagcacgtcggcatcgcctgagaagaactgaaatcgcccgttcaccgctttaagtagaagcaaaagagcatctcatcggaatggctcagaggattatatattgatcggcctatttgagaactacacgtttacgcacaaaagaaataaacacatctcaatgcaaaa

SEQ ID NO: 5 - DNA, 1443 bp, Cucumis sativus
  misc_feature 1..1443: note = Modified CDS of CsDCAF8
  source 1..1443: mol_type = genomic DNA
atgaacaagagggctagaattgacgattccaataaggcagtggtcaatgtctggaagcgggaggttggagagctttccactaggaattttgcccaccgtcttggtgctttggaggatctcgtgctccgacttgatctttacaagaaactggaaaggcacaaaggttgtgtgaacaccgtgagctttaatgctgctggtgatattttagtttcaggctctgatgataggcgggtgattctctggaattgggaaaccgggcgtgtcaagctctcttttcagtctggtcacaataacaatgtttttcaagcaaaaatcatgccttacacggatgatcaaagcattgtgacttgtgctgcagatgggcaggtgaggcatgctcagattctgaatagtgggcatgtggaaacttcactactcggcaatcatctagggcgagctcataagttggccattgaacctggtagtccttatatgttctatacttgtggtgaagatggattggtgcaacgttttgatctaagaactggggatgccgttgagctgttcacttgccaatcagttgacaatagggcaggttacatgtcatcgattgagcttaatgcaattgtgattgatcccagaaatccaaatctctttgttgttgctggttcagatgagtatgcacgattgtatgatattcgcaagtctggggaggatggttccactgattttggtcaactggcagactgctattgcccccctcacttgattggtgatgagcaagtgggaataacaggtctagccttctcagagctcagtgagctccttgtctcatacaatgatgaatccatttatctctttaatagggatatgggtttgggcccaaacccaaatccagtccctccactatctctgagtagtgatgcaagtgaaatgggagctgaaaacgttgacaatggtattccacaagtttataaaggacatagaaactgtgagacagttaaaggggtcaatttttttggacctaaatgtgagtatgtcgttagtggttctgattgtggcagaatatttatatggaggaaaaaaggtggagatctaatccgtgtcatggaagctgacgaggatgtcgtgaactgcatcgagccccatcctcatatgactatgcttgctagtagtggaattgaaagtgatgtcaagatatggactccaaaggcccatgaaagagctactcttcctgagaaaattgaacagaaaccaaagcccagaggatggatgcaccgcttagccttaccggaggaactgatgatgcaactgttttcactgcgaagacagacttcgagccctgaaagtggaagagaacctcctactgtaagtagggaacttctagagttgataaggacattcaacaatggcaacagcgatgactcttcagattataatgatgatagtggtgaagatgattga

SEQ ID NO: 6 - protein, 480 aa, Cucumis sativus
  REGION 1..480: note = Modified protein sequence of CsDCAF8
MNKRARIDDSNKAVVNVWKREVGELSTRNFAHRLGALEDLVLRLDLYKKLERHKGCVNTVSFNAAGDILVSGSDDRRVILWNWETGRVKLSFQSGHNNNVFQAKIMPYTDDQSIVTCAADGQVRHAQILNSGHVETSLLGNHLGRAHKLAIEPGSPYMFYTCGEDGLVQRFDLRTGDAVELFTCQSVDNRAGYMSSIELNAIVIDPRNPNLFVVAGSDEYARLYDIRKSGEDGSTDFGQLADCYCPPHLIGDEQVGITGLAFSELSELLVSYNDESIYLFNRDMGLGPNPNPVPPLSLSSDASEMGAENVDNGIPQVYKGHRNCETVKGVNFFGPKCEYVVSGSDCGRIFIWRKKGGDLIRVMEADEDVVNCIEPHPHMTMLASSGIESDVKIWTPKAHERATLPEKIEQKPKPRGWMHRLALPEELMMQLFSLRRQTSSPESGREPPTVSRELLELIRTFNNGNSDDSSDYNDDSGEDD

SEQ ID NO: 7 - DNA, 201 bp, Cucumis sativus
  misc_feature 1..201: note = Marker sequence 1
  source 1..201: mol_type = genomic DNA
yccccagacttgcgaatatcatacaatcgtgcatactcatctgaaccagcaacaacaaagagatttggatttctgggatcaatcacaattgcattaagctcaatcgatgacatgtaacctgccctattgtcaactgattggcaagtgaacagctcaacggcatccccagttcttagatcaaactataaaattagtaaagtc

SEQ ID NO: 8 - DNA, 201 bp, Cucumis sativus
  misc_feature 1..201: note = Marker sequence 2
  source 1..201: mol_type = genomic DNA
yccccagacttgcgaatatcatacaatcgtgcatactcatctgaaccagcaacaacaaagagatttggatttctgggatcaatcacaattgcattaagctgaatcgatgacatgtaacctgccctattgtcaactgattggcaagtgaacagctcaacggcatccccagttcttagatcaaactataaaattagtaaagtc